(19) United States
(12) Patent Application Publication
(10) Pub. No.: US 2007/0061899 A1
(43) Pub. Date: Mar. 15, 2007

(54) Transgenic mammal with constitutive, tissue-specific cytokine expression and use as a cancer model

(75) Inventor: Timothy C. Wang, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/404,226

(22) Filed: Apr. 13, 2006

Related U.S. Application Data
(60) Provisional application No. 60/670,911, filed on Apr. 13, 2005.
(60) Provisional application No. 60/705,429, filed on Aug. 4, 2005.

(51) Int. Cl. A01K 67/027

(52) U.S. Cl. 800/6; 800/18

(57) ABSTRACT

The invention is directed to a transgenic non-human mammal with constitutive, tissue-specific cytokine expression and the use of the transgenic mammal as a model for spontaneous development of cancer. Particularly, the invention provides for a transgenic mouse where specific expression of interlueukin-1β in the stomach results in the development of gastric cancer. The invention also encompasses methods using the transgenic mammals for screening compounds for their ability to modulate the development, growth or progression of cancer.

This application claims priority to U.S. Application No. 60/670,911, filed Apr. 13, 2005, and U.S. Application No. 60/705,429, filed Aug. 4, 2005, both of which are hereby incorporated by reference in their entireties.

The invention disclosed herein was made with U.S. Government support under NIH Grant No. CA93405 from the NCI. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### FIELD OF THE INVENTION

This invention is directed toward a transgenic mammal with constitutive, tissue-specific cytokine expression and use of the transgenic mammal as a cancer model. For example, the invention provides for a transgenic mouse wherein constitutive expression of IL-1β in the stomach results in the spontaneous development of gastric cancer in the mouse.

### BACKGROUND OF THE INVENTION

Gastric cancer, one of most common cancers in the world, is a result of interaction between genetic factors of the host together with dietary and other factors in the environment. Epidemiological studies on Northern Chinese and American Japanese in Hawaii lent strong support to the effects of lack of fresh fruit and vegetable, smoking, and consumption of salty food in the development of gastric cancer. In the last decade, it has been proposed that Helicobacter pylori plays a pivotal role in triggering chronic inflammation of the stomach leading to stepwise development of the malignancy.

### SUMMARY OF THE INVENTION

The invention provides for a transgenic non-human mammal whose somatic and germ cells comprise (a) a DNA segment encoding a human cytokine operably linked to a DNA segment encoding a secretory signal sequence; and (b) a constitutively active tissue-specific promoter wherein (a) is operably linked to (b). In one embodiment, the tissue for which the promoter is specific comprises breast tissue, colon tissue, pancreas tissue, lung tissue, ovary tissue, cervical tissue, uterine tissue, bone tissue, stomach tissue, gastric tissue, testicular tissue, prostate tissue, skin tissue, esophagus tissue, liver tissue, kidney tissue, bladder tissue, or any combination thereof. In another embodiment, the cytokine comprises interleukin-1 beta, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, WFN-γ, IFN-α/β, SDF-1/CXCL12, MIP-1α/CCL3, MCP-1/CCL2, SCF, or any combination thereof. In a preferred embodiment, the linked DNA segments are integrated into the mammal's genome.

The invention also provides for a nucleic acid comprising (a) a tissue-specific promoter operably linked to a DNA segment encoding a secreted human cytokine, or a fragment thereof; and (b) a polyadenylation signal, wherein (a) and (b) are operably linked and wherein the nucleic acid is capable of producing expression of the cytokine in the specific tissue in a transgenic mammal.

Another aspect of the invention provides for a transgenic non-human mammal whose somatic and germ cells comprise (a) a DNA segment encoding human interleukin-1β operably linked to a DNA segment encoding a secretory signal sequence; and (b) a constitutively active parietal cell-specific promoter, wherein (a) is operably linked to (b). In one embodiment, of the DNA segment results in gastritis, dysplasia, spontaneous development of gastric cancer, or any combination thereof in the mammal. In a particular embodiment, the promoter comprises a mouse H/K-ATPase promoter, or a functional fragment thereof.

Further provided for by the present invention is a transgenic non-human mammal whose somatic and germ cells comprise (a) a DNA segment encoding human interleukin-1β operably linked to a DNA segment encoding a secretory signal sequence; and (b) a constitutively active pancreas-specific promoter, wherein (a) is operably linked to (b). In an embodiment, expression of the DNA segment results in pancreatic interepithelial neoplasia, spontaneous development of pancreatic cancer, or both, in the mammal. In another embodiment, the promoter comprises a rat elastase promoter, or a functional fragment thereof.

In a preferred embodiment of the invention, the secretory signal sequence comprises a signal sequence from an IL-1 receptor antagonist gene, or a fragment thereof.

In the practice of the invention, the mammal is a mouse.

Provided for by one aspect of the invention is a transgenic non-human mammal whose somatic and germ cells comprise a DNA segment encoding a human cytokine operably linked to a constitutively active stomach-specific H/K-ATPase promoter, K19 promoter, TFF1 promoter, TFF2 promoter, FOXa3/HNF3γ promoter, or a functional fragment thereof, wherein expression of the DNA segment results in spontaneous development of cancer in the stomach of the mammal.

Provided for by another aspect of the invention is a transgenic non-human mammal whose somatic and germ cells comprise a DNA segment encoding a human cytokine operably linked to a constitutively active lung-specific Clara cell secretory protein promoter, surfactant protein C promoter, or a fragment thereof, wherein expression of the DNA segment results in spontaneous development of cancer in the lungs of the mammal.

Provided for by yet another aspect of this invention is a transgenic non-human mammal whose somatic and germ cells comprise a DNA segment encoding a human cytokine operably linked to a constitutively active breast-specific mouse mammary tumor virus (MMTV) promoter, whey acidic protein promoter, or a fragment thereof, wherein expression of the DNA segment results in spontaneous development of cancer in a breast of the mammal.

The present invention provides for a transgenic non-human mammal whose somatic and germ cells comprise a DNA segment encoding a human cytokine operably linked to a constitutively active pancreas-specific Pdx-1 promoter, insulin promoter, phosphoglycerate kinase promoter, elastase promoter, or a fragment thereof, wherein expression of the DNA segment results in spontaneous development of cancer in the pancreas of the mammal.

The present invention also provides for a transgenic non-human mammal whose somatic and germ cells comprise a DNA segment encoding a human cytokine operably linked to a constitutively active skin-specific keratin promoter, or a fragment thereof, wherein expression of the DNA segment results in spontaneous development of cancer in the skin of the mammal.

The present invention further provides for a transgenic non-human mammal whose somatic and germ cells comprise a DNA segment encoding a human cytokine operably linked to a constitutively active esophagus-specific EBV ED-L2 promoter, or a fragment thereof, wherein expression of the DNA segment results in spontaneous development of cancer in the esophagus of the mammal.

One aspect of this invention provides for a transgenic non-human mammal whose somatic and germ cells comprise a DNA segment encoding a human cytokine operably linked to a constitutively active liver-specific major urinary protein (MUP) promoter, albumin promoter, or a fragment-thereof, wherein expression of the DNA segment results in spontaneous development of cancer in the liver of the mammal.

Another aspect of this invention provides for a transgenic non-human mammal whose somatic and germ cells comprise a DNA segment encoding a human cytokine operably linked to a constitutively active colon-specific villin promoter, FABP-TS4 promoter, or a fragment thereof, wherein expression of the DNA segment results in spontaneous development of cancer in the colon of the mammal.

A further aspect of the invention provides for a transgenic non-human mammal whose somatic and germ cells comprise a DNA segment encoding a human cytokine operably linked to a prostate-specific constitutively active cryptdin-2 promoter, prostate-specific antigen (PSA) promoter, C(3)1 promoter, prostate secretory protein of 94 amino acids (PSP94) promoter, or the probasin promoter, or a fragment thereof, wherein expression of the DNA segment results in spontaneous development of cancer in the prostate of the mammal.

Yet another aspect of the invention provides for a transgenic non-human mammal whose somatic and germ cells comprise a DNA segment encoding a human cytokine operably linked to a constitutively active ovarian-specific promoter (OSP-1), or a fragment thereof, wherein expression of the DNA segment results in spontaneous development of cancer in one or both ovaries of the mammal.

In an aspect of this invention, is a transgenic non-human mammal whose somatic and germ cells comprise a DNA segment encoding a human cytokine operably linked to a constitutively active kidney-specific uromodulin promoter, Tamm-Horsfall protein (THP) promoter, or type 1 gamma-glutamyl transpeptidase promoter, or a fragment thereof, wherein expression of the DNA segment results in spontaneous development of cancer in one or both kidneys of the mammal.

The invention also provides for a transgenic non-human mammal whose somatic and germ cells comprise a DNA segment encoding a human cytokine operably linked to a constitutively active bladder-specific uroplakin promoter or urohingin promoter, or a fragment thereof, wherein expression of the DNA segment results in spontaneous development of cancer in the bladder of the mammal.

Additionally provided for by the invention is a transgenic non-human mammal whose somatic and germ cells comprise a DNA segment encoding a human cytokine operably linked to a constitutively active uterus-specific uteroglobin promoter, or a fragment thereof, wherein expression of the DNA segment results in spontaneous development of cancer in the uterus of the mammal.

In accord with this invention, the cytokine expressed by the transgenic mammal comprises an inflammatory cytokine. In preferred embodiments, the cytokine comprises TNF-A, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, WFN-γ, IFN-α/β, SDF-1/CXCL12, MIP-1α/CCL3, MCP-1/CCL2, SCF, or any combination thereof. In a particular embodiment, the cytokine comprises a secreted form of human interleukin-1β.

Also in accordance with the present invention is a cell from the non-human transgenic mammal provided for by the invention.

One aspect of this invention provides for a method for identifying whether a test compound is capable of treating cancer, the method comprising (a) administering an effective amount of a test compound to a transgenic non-human mammal of provided for by the invention; (b) measuring progression of cancer in the transgenic non-human mammal of (a); and (c) comparing the progression of cancer measured in (b) to progression of cancer measured in a sibling of the transgenic non-human mammal, wherein the sibling was not administered the test compound, and wherein a decrease in progression of cancer in the non-human mammal of (a) compared to (b) indicates that the test compound is capable of treating cancer. In accordance with this method, the transgenic non-human mammal has cancer. In the practice of this method, a decrease comprises an arrest, delay or reversal in progression of cancer and the measuring comprises a histological assessment, an assessment of alterations in the mammal's weight and activity, non-invasive imaging, an assessment of serum biomarkers, or any combination thereof.

Another aspect of the invention provides for a method for identifying whether a test compound is capable of preventing or delaying the development of cancer, the method comprising (a) administering an effective amount of a test compound to a transgenic non-human mammal of the invention, wherein the transgenic non-human mammal does not have cancer; (b) measuring development of cancer in the transgenic non-human mammal of (a); (c) comparing the development of cancer measured in (b) to development of cancer measured in a sibling of the transgenic non-human mammal, wherein the sibling was not administered the test compound, and wherein inhibition of or a delay in the development of cancer in the non-human mammal of (a) compared to (b) indicates that the test compound is capable of preventing or delaying the development of cancer.

Also provided for by the present invention is a method for identifying whether a test compound is a carcinogen, the method comprising (a) administering to a transgenic non-human mammal of the invention or exposing a transgenic non-human mammal of the invention to an effective amount of a test compound, wherein the transgenic non-human mammal does not have cancer; (b) measuring development of cancer in the transgenic non-human mammal of (a); (c) comparing the development of cancer measured in (b) to development of cancer measured in a sibling of the transgenic non-human mammal, wherein the sibling was not administered the test compound or exposed to the test compound, and wherein earlier development of cancer in the non-human mammal of (a) compared to (b) indicates that the test compound is a carcinogen.

In accordance with the methods of the presents invention, the cancer comprises a breast cancer, a colon cancer, a pancreatic cancer, a lung cancer, an ovarian cancer, a cervical cancer, a uterine cancer, a bone cancer, a stomach cancer, a gastric cancer, a testicular cancer, a prostate cancer, a skin cancer, an esophageal cancer, a liver cancer, a kidney cancer, a bladder cancer, a lymphoma, or any combination thereof.

### BRIEF DESCRIFTION OF THE FIGURES

FIGS. 1A-1D. Human IL-1β was specifically overexpressed in gastric mucosa of IL-1β transgenic mice. FIG. 1A. The construct of pBS/HKATPase/β globin/IL-1β contains the mouse H⁺/K⁺-ATPaseβ subunit gene and secreted form of hIL-1β cDNA. The construct was injected into the oocytes. FIG. 1B. The concentration of hML-1β in the stomach and spleen tissues. The 5 founders were sacrificed and stomach tissue was collected and lysed. The concentration of hIL-1β in the stomach-tissues was determined using hML-1β ELISA. FIG. 1C. The concentration of hML-1β in the stomach and serum were measured. Line 19 and line 42 transgenic mice and control mice at the age of 4 months and more than 12 months were sacrificed. Serum and stomachs were collected. The concentrations of hIL-1β, were determined by using hML-1β ELISA kit. (Control mice n=6; Line 19 n=10; Line 42 n=12 at different ages, respectively. Line 19 vs. control group, p<0.01). FIG. 1D. The transgenic mice and control mice were sacrificed and stomach and spleen were collected. The mRNA of stomach and spleen was abstracted by TRIZOL kit. The expression of mouse Il-1β mRNA in stomach and spleen tissues was determined by Polymerase chain reaction (PCR).

FIGS. 2A-2B. The II-1β transgenic mice developed gastric inflammation and dysplasia (carcinoma) in the stomach. FIG. 2A. The larger size of stomach in the IL-1β transgenic mice. The transgenic mice and control mice at the age of 12-20 months were sacrificed, and stomach photographs were taken. The typical stomach photograph from 16 months old mice were showed. FIG. 2B. Sections of stomach from 12-month-old transgenic and control mice were stained with H & E. The morphologic analysis of the gastric mucosa in IL-1β mice demonstrating hyperplasia, inflammatory changes. The mucosa of IL-1β transgenic mice was severely hyperplasia in line 19 mice with inflammatory foci and moderate hyperplasia line 42 mice. (Original magnification: Upper line, 40×; lower line, 200×).

FIGS. 3A-3B. The IL-1β transgenic mice developed dysplasia and carcinoma in stomach. FIG. 3A. The dysplasia morphologic changes in the gastric mucosa of IL-1β mice. Sections of stomach from 16-month-old transgenic and control mice were stained with H & E. The mucosa of Il-1β transgenic mice was severe dysplasia in Line 19 mice with inflammatory foci and moderate dysplasia Line 42 mice. (Original magnification: Upper line, 40×; lower line, 200×). FIG. 3B. The line 19 IL-1β transgenic mice developed stomach cancer. The line 19 male mice at the age of 14 months old were sacrificed and stomach pictures were taken. The tumors were obviously observed in the stomach. Sections of stomach were stained with H & E. The mucosa of transgenic mice exhibited a typical the adenomatous structure and high degree dysplastic morphological changes (carcinogenesis). (Original magnification: middle panel, 40×; Third panel, 200×).

FIGS. 4A-4C. Overexpression of IL-1β, inhibited gastric acid secretion and loss of parietal cells. FIG. 4A. The pH values in gastric juice. IL-1β, and control mice at ages of 12 months were selected for determination of acid secretion via pyloric ligation technique. Gastric juice was collected and measured with a pH meter by 0.01 NNaOH titration. The data represented the average of 5˜6 mice. P<0.05, compared with control wild type mice of the same age. FIG. 4B. The concentration of gastrin in the serum. The serum was collected when mice—were sacrificed and the serum level of gastrin was determined by radioimmunoassay (RIA). The data represents the average results from 5-6 mice. P<0.05, compared with control wild type mice of the same age. FIG. 4C. Loss of parietal cells in transgenic mice. Stomach tissues from transgenic mice and control were collected, and tissue sections of formalin-fixed. The sections were stained by immunohistochemical staining with H/K-ATPaseβ subunit antibody. (Original magnification 150×). The brown staining cells indicate the parietal cells.

FIG. 5. Cell proliferation and apoptosis in stomach tissues. The section from transgenic mice and control were stained with Ki-67 antibody for cell proliferation (upper lane), or TUNEL staining for apoptosis (lower lane). Images were representative fields of view from stomach tissues of mice at the age 12 months. (Original magnification 150×). Arrows showed the brown staining cells (Ki-67 positive cells, upper lane) and apoptotic cells (lower lane).

FIGS. 6A-6E. Overexpression of IL-1β increased the production of proinflammatory cytokines. FIG. 6A. The increase in mRNA expression of proinflammatory cytokines. The total mRNA was abstracted from stomach and spleen tissues of transgenic and control mice. The mRNA expression of cytokines was determined by RT-PCR. The presented date were representative results from transgenic and control mice at the age 12 months. FIGS. 6B-6C. The increase in the levels of mouse TNF-α and IL-6 in the serum and stomach tissues. The serum was collected, and the stomachs of transgenic mice were collected and lysed. The concentrations of mouse TNF-α and IL-6 in the stomach were determined by ELISA method. (Line 19, n=15; Line 42 n=12, control mice n=6), *P<0.05, compared with control mice. FIGS. 6D-6E. The increase in the levels of mouse IL-1RA in the serum and stomach tissues. The concentrations of mouse IL-1RA were determined by ELISA method. (Control mice n=6; Line 42 n=12; Line 19, n=15). *P<0.05, compared with control mice.

FIGS. 7A-7B. The IL-1β, transgenic mice caused the inflammatory response in the spleen tissues. FIG. 7A. The splenomegaly in transgenic mice. The transgenic mice and control mice at the age of 12-20 months were sacrificed, and stomach photographs were taken. The typical spleen photograph from 16 months old mice were showed. FIG. 7B. The pathological change and the inflammatory infiltration in the spleen. The Sections of spleen from 16-month-old transgenic and control mice were stained with H & E. The morphologic analysis in the spleen demonstrates the inflammatory infiltration of leukocytes. (Original magnification: 150×).

FIGS. 8A-8D. Overexpression of IL-1β increased the infiltration of T cell in stomach tissues. T and B lymphocytes infiltration in mucosa and epithelial area of stomach tissues of the control mice (FIG. 8A) and IL-1β mice (FIG. 8B). Froze sections of stomach were stained with FITC-labelled CD3, CD4, CD8 and CD19. Sections were double stained with DAPI to visualize stomach morphology. Fluorescence images were captured on Fluorescence microscope with identical exposure times. Sections were captured with a with a ×200 objective. FIG. 8C. Quantification of T and B lymphocytes in the stomach tissues. The averages were obtained from three sections at five random views for each section with 200 magnification. FIG. 8D. NF-κB activation in the stomach tissues of IL-1β transgenic mice. The sections of stomach from 12-month-old transgenic and control mice were stained with NF-κB antibody. (Original magnification: 100×, small picture 400×).

FIG. 9. Helicobacter felis infection accelerates the development of gastritis and carcinoma in IL-1β mice. The sections of stomach were from 6-month old transgenic mice and control mice infected with H. felis for 4 months. The sections were stained with H & E.

FIGS. 10A-10C. The increase in expression of TFF2 in the stomach tissues of IL-1β mice FIG. 1A. Expression of TFF2 in stomach tissues from control mice, IL-1β line 42 and line 19 mice. Stomach sections were immunostained with anti-TFF2 antibody. Arrows, TFF2-positive cells in the neck; arrowheads, TFF2-positive cells in the endothelial cells. Note that most mucous metaplastic cells express both TFF2 in the stomach of IL-1β mice. FIG. 10B. Quantity of TFF2-positive cells in mucous metaplastic area of stomach. Score Criteria were defined as the percentage of TFF2 positive cells in glandular tissues: Score 0: None; 1: <25%; 2: 25%-50% 3: 50%-75%; 4: >75%. Control mice n=3; line 42 n=8; line 19, n=12. FIG. 10C. mRNA expression of TFF2 in the stomach tissues was determined by real-time PCR. The present result was the relative mRNA expression of TFF2 of IL-1β mice compared with that of control mice. Control mice n=3; line 42 n=8; line 19, n=8.

FIGS. 11A-11B. Overexpression of IL-1β increased angiogenesis in stomach tissues. FIG. 11A. The expression of CD34, Cox-2 and VEGF in the stomach tissues of IL-1β mice and control mice. The stomach sections from control mice (left lane), IL-1β line 42 mice (middle lane) and line 19 mice (left lane) were immunostained with anti-CD34 (upper lane), Cox-2 (middle lane) and VEGF (lower line) antibodies. Photographs were taken from representative sections with a ×200 objective. FIG. 11B. The protein expression of Cox-2, VEGF and MMP-9 were determined by Western blot in the stomach tissues from 2 representative control mice, IL-1β line 42 mice and line 19 mice.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

The present invention is directed to a transgenic non-human mammal with constitutive, tissue-specific cytokine expression and use of the transgenic mammal, such as for a cancer model. In one embodiment, the invention encompasses an IL-1β transgenic mouse, wherein the constitutive transgene expression is directed specifically to the parietal tissue (stomach), and wherein the transgenic mammal develops gastric cancer. The present invention provides for a human IL-1β (hIL-1β) transgenic mouse with targeted expression of IL-1β to the stomach. Interleukin-1 beta has been shown to be relevant in the development of cancer in human patients. The IL-1β transgenic mouse develops spontaneous inflammation, dysplasia and carcinoma in the stomach. The mechanisms of spontaneous development of gastric cancer involve inhibition of gastric acids, proinflammatory cytokines, and dysregulation of immunity. IL-1β plays a pivotal role in development of gastric carcinogenesis, thereby representing a therapeutic target for prevention and treatment of stomach cancer.

The biological effects of IL-1β as a potent proinflammatory cytokine and inhibitor of acid suppression fits with the proposed model. IL-1β, induced by H. pylori infection, is known to be a strong proinflammatory cytokine as well as a strong inhibitor of acid secretion in the stomach. IL-1 gene cluster (IL-1β encoding IL-1β and IL-1β encoding the IL-1β receptor antagonist) has a number of functionally relevant polymorphisms that may correlate with high or low IL-1β production. The association between IL-1 genotypes and gastric cancer was confined to the intestinal type but not diffuse type of gastric cancer. These data showed that Il-1β polymorphisms are associated with an increased risk of gastric cancer development. However, direct evidence in which IL-1β induces stomach carcinogenesis remains to be obtained. Therefore, a transgenic mouse with targeted expression of IL-1β in the parietal cells of the stomach has been generated and was found to spontaneously develop gastric carcinoma in a manner akin to the development of cancer in humans.

The transgenic mammals provided for by the invention can be used as an accelerated model of cancer. The transgenic mammal can also be used as a physiological model of cancer, for example gastric cancer, that can be further used for imaging and screening drugs or other compounds. The present invention provides for a cancer model based on human pathophysiology (i.e., spontaneous development of cancer).

This invention provides for platform transgenic mammals for a new model of cancer development, especially inflammation-related cancers. There are presently no reliable animal models to study this mode of cancer development. The transgenic mammals provided for by this invention fill this void in cancer research. The overexpression of a cytokine in the inventive mammals mimics the physiology of inflammation in humans. The transgenic mammals spontaneously develop cancer specifically in the tissue to which cytokine overexpression is directed. Animal models of cancer currently available develop cancer due to the overexpression of an oncogene or a transgene, resulting in the transformation of cells to which expression is directed. In contrast, in the model of the present invention, cells do not develop a cancer phenotype due to the expression of a cancer-promoting transgene. Rather, cells become spontaneously transformed due to the specific environment created by cytokine overexpression. Thus, an increase in extracellular cytokine concentration, as would be present in a state of chronic inflammation, results in the spontaneous development of cancer. Therefore, the transgenic mammals of the present invention provide for more accurate animal models of the development of cancer in humans. Animal models of this type are needed in research institutes to study mechanisms underlying the development, growth and progression of cancer; these models are also in demand at pharmaceutical companies investigating potential therapeutic effects of compounds on the development, growth and progression of cancer.

Transgenic Non-Human Mammals

Transgenic mammals are produced by introducing into the genome of the mammal a transgene DNA construct designed to target the expression of a cytokine to cells of a specific tissue. The transgene is introduced into the mammal, or an ancestor of the mammal, at an embryonic stage according to standard transgenic techniques. For example, one technique for transgenically altering a mammal is to microinject the transgene DNA construct into the pronuclei of one or more fertilized mammalian eggs. The eggs are transferred to a foster female, where the eggs are allowed to develop to full term. U.S. Pat. Nos. 4,736,866, 5,087,571 and 5,925,803 provide detailed explanations for the production and analysis of transgenic mice.

Provided for by this invention is a transgenic non-human mammal expressing a cytokine operably linked to a tissue-specific promoter, and use of the transgenic mammal as a cancer model. Tissue-specific cytokine expression in transgenic mice has been in numerous studies, however there are no reports of tissue-specific cytokine overexpression leading to the development of cancer in the transgenic mice. For example, transgenic mice expressing IL-6, IL-11 and IL-10 in the lung have been engineered, but cancer development was not reported in any of the mice (Kuhn et al., Am J Respir Cell Mol Biol 22:289-295 (2000); Lee et al., J Biol Chem 277:35466-35474 (2002); Wang et al., J Immunol 165:2222-22231 (2000)). IL-10 and IL-8 have been expressed specifically in the intestine of transgenic mice, but the studies did not report that cytokine overexpression resulted in cancer (DeWinter et al., Gasteroenterology 122:1829-1841 (2002); Wen et al., J Immunol 166:7290-7299 (2001)). This invention provides for the discovery of spontaneous cancer development in transgenic non-human mammals expressing a cytokine linked to a tissue-specific promoter. The invention provides for transgenic non-human mammals with tissue-specific cytokine expression and the use of the transgenic mammals as a model for spontaneous development of cancer.

The present invention provides for a transgenic non-human mammal whose somatic and germ cells comprise (a) a DNA segment encoding a human cytokine operably linked to a DNA segment encoding a secretory signal sequence; and (b) a constitutively active tissue-specific promoter wherein (a) is operably linked to (b). In one embodiment, the tissue for which the promoter is specific comprises breast tissue, colon tissue, pancreas tissue, lung tissue, ovary tissue, cervical tissue, uterine tissue, bone tissue, stomach tissue, gastric tissue, testicular tissue, prostate tissue, skin tissue, esophagus tissue, liver tissue, kidney tissue, bladder tissue, or any combination thereof. In another embodiment, the cytokine comprises interleukin-1 beta, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF or any combination thereof. In a preferred embodiment, the linked DNA segments are integrated into the mammal's genome.

Another aspect of this invention provides for a transgenic non-human mammal whose somatic and germ cells comprise (a) a DNA segment encoding human interleukin-1β operably linked to a DNA segment encoding a secretory signal sequence; and (b) a constitutively active parietal cell-specific promoter, wherein (a) is operably linked to (b). In one embodiment, expression of the DNA segment results in gastritis, dysplasia, spontaneous development of gastric cancer, or any combination thereof in the mammal. In another embodiment, the promoter comprises mouse H/K-ATPase promoter or a functional DNA fragment thereof. Preferably, the H/K-ATPaseβ subunit promoter is used to direct transgene expression, however the H/K-ATPase alpha subunit is also stomach-specific and can be used within the context of this invention. In another embodiment, the secretory signal sequence comprises a signal sequence from an EL-1 receptor antagonist gene, or a fragment thereof. In a preferred embodiment, the mammal is a mouse. Also within the context of this invention is the use of a transgenic non-human mammal with stomach-specific expression of IL-1β, as a model for B-cell lymphoma.

Another aspect of this invention provides for a transgenic non-human mammal whose somatic and germ cells comprise (a) a DNA segment encoding human interleukin-1β operably linked to a DNA segment encoding a secretory signal sequence; and (b) a constitutively active pancreas-specific promoter, wherein (a) is operably linked to (b). In one embodiment, expression of the DNA segment results in pancreatic intraepithelial neoplasia (PIN), spontaneous development of pancreatic cancer in the mammal, or both. In another embodiment, the promoter comprises the rat elastase promoter or a functional DNA fragment thereof. In another embodiment, the secretory signal sequence comprises a signal sequence from an IL-1 receptor antagonist gene, or a fragment thereof. In a preferred embodiment, the mammal is a mouse.

This invention is also directed to a transgenic non-human mammal whose somatic and germ cells comprise a DNA segment encoding a human cytokine operably linked to a constitutively active H/K-ATPase promoter, K19 promoter, metallothionein/MT-1 promoter, TFF1 promoter, TFF2 promoter, FOXa/HNF3γ promoter, or a functional fragment thereof, wherein expression of the DNA segment results in spontaneous development of cancer in the stomach of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

One aspect of the present invention provides for a transgenic non-human mammal whose somatic and germ cells comprise a DNA segment encoding a human cytokine operably linked to a constitutively active promoter selected from the group consisting of: a Clara cell secretory protein promoter, a surfactant protein C promoter, or a fragment thereof, wherein expression of the DNA segment results in spontaneous development of cancer in the lungs of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

Another aspect of this invention provides for a transgenic non-human mammal whose somatic and germ cells comprise a DNA segment encoding a human cytokine operably linked to a constitutively active mouse mammary tumor virus (MMTV) promoter, whey acidic protein promoter, or a fragment thereof, wherein expression of the DNA segment results in spontaneous development of cancer in the breasts of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, EFN-α/β, SDF-1/CXCL12, MIP1α/CCL3, MCP-1/CCL2, SCF, or any combination thereof.

The present invention also encompasses a transgenic non-human mammal whose somatic and germ cells comprise a DNA segment encoding a human cytokine operably linked to a constitutively active Pdx-1 promoter, insulin promoter, phosphoglycerate kinase promoter, elastase promoter, or a fragment thereof, wherein expression of the DNA segment results in spontaneous development of cancer in the pancreas of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, EFN-α/β, SDF-1/CXCL12, MIP1β/CCL3, MCP-1/CCL2, SCF, or any combination thereof.

In another aspect, the invention provides for a transgenic non-human mammal whose somatic and germ cells comprise a DNA segment encoding a human cytokine operably linked to a constitutively active keratin promoter, K14 promoter, or a fragment thereof, wherein expression of the DNA segment results in spontaneous development of cancer in the skin of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

The invention also provides for transgenic non-human mammal whose somatic and germ cells comprise a DNA segment encoding a human cytokine operably linked to a constitutively active EBV ED-L2 promoter, or a fragment thereof, wherein expression of the DNA segment results in spontaneous development of cancer in the esophagus of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-S, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

This invention is also directed to a transgenic non-human mammal whose somatic and germ cells comprise a DNA segment encoding a human cytokine operably linked to a constitutively active mouse major urinary protein (MUP) promoter, albumin promoter, or a fragment thereof, wherein expression of the DNA segment results in spontaneous development of cancer in the liver of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

The invention additionally provides for a transgenic non-human mammal whose somatic and germ cells comprise a DNA segment encoding a human cytokine operably linked to a constitutively active villin promoter, FABP-TS4 promoter, or a fragment thereof, wherein expression of the DNA segment results in spontaneous development of cancer in the colon of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

Also provided for by the present invention is a transgenic non-human mammal whose somatic and germ cells comprise a DNA segment encoding a human cytokine operably linked to a constitutively active cryptdin-2 promoter, prostate-specific antigen (PSA) promoter, C(3)1 promoter, prostate secretory protein of 94 amino acids (PSP94) promoter, or the probasin promoter, or a fragment thereof, wherein expression of the DNA segment results in spontaneous development of cancer in the prostate of the mammal. In one embodiment, the cytokine comprises an inflammatory cytokine. In another embodiment, the cytokine comprises a secreted form of interleukin-lo. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

Further provided for by this invention is a transgenic non-human mammal whose somatic and germ cells comprise a DNA segment encoding a human cytokine operably linked to a constitutively active ovarian-specific promoter (OSP-1), or a fragment thereof, wherein expression of the DNA segment results in spontaneous development of cancer in one or both ovaries of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

In another aspect, the invention covers a transgenic non-human mammal whose somatic and germ cells comprise a DNA segment encoding a human cytokine operably linked to a constitutively active uromodulin promoter, Tamm-Horsfall protein (THP) promoter, or type 1 gamma-glutamyl transpeptidase promoter, or a fragment thereof, wherein expression of the DNA segment results in spontaneous development of cancer in one or both kidneys of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

An additional aspect of this invention provides for a transgenic non-human mammal whose somatic and germ cells comprise a DNA segment encoding a human cytokine operably linked to a constitutively active uroplakin promoter or urohingin promoter, or a fragment thereof, wherein expression of the DNA segment results in spontaneous development of cancer in the bladder of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

In a further aspect, this invention provides for a transgenic non-human mammal whose somatic and germ cells comprise a DNA segment encoding a human cytokine operably linked to a constitutively active uteroglobin promoter, or a fragment thereof, wherein expression of the DNA segment results in spontaneous development of cancer in the uterus of the mammal. In one embodiment, the cytokine comprises an inflammatory cytokine. In another embodiment, the cytokine comprises a secreted form of interleukin-1β. In yet another embodiment, the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

Cytokines and Chemokines

The invention provides for transgenic non-human mammals expressing DNA segments encoding a cytokine or a chemokine. In accord with the invention, the cytokine or chemokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1/CCL3, MCP-1/CCL2, SCF, or any combination thereof. In a preferred aspect of the invention, the cytokine is a secreted form of IL-1β. In addition to the foregoing list, those skilled in the art will appreciate other cytokines and chemokines that fall within the scope of this invention.

Interleukin-1β

Interleukin-1 is proinflammatory peiotropic cytokine that affects mainly hematopoiesis, inflammation, immunity and tumorigenicty (Apte, R. N., et al. Semin Cancer Biol 2002;12:277˜290; Dinarello C. A. Blood 1996; 87: 2095-2147). The properties of IL-1 stem from its ability to induce the synthesis of cytokines, chemokines, proinflammatory molecules, and the expression of adhesion molecules (Apte, R. N., et al. Semin Cancer Biol 2002;12:277˜290). The IL-1 gene family consists of two major agonistic molecules, namely IL-1α and IL-1β, and one antagonistic cytokine, the IL-1R antagonist (IL-1Ra) (P. E. Auron, Cytokine Growth Factor Rev. 1998; 9: 221-237). IL-1Ra binds to the IL-1 receptor type I without transmitting an activation signal and, thus, represents a physiological inhibitor of IL-1 activity. IL-1α and IL-1β bind to the same receptors, and there are no significant differences in the spectrum of activities induced by recombinant IL-1α and IL-1β by using in vitro and in vivo assays, including injection into humans (Apte, R. N., et al. Semin Cancer Biol 2002; 12:277˜290).

IL-1β is involved in angiogenesis, tumorigenesis and invasion. IL-1β markedly induced angiogenesis in vitro and in vivo by upregulation of expression of various prostanoids (Vidal-Vanaclocha et al., Proc Natl Acad Sci USA. 2000; 97:734-9.). In microenvironmental stroma cells and in malignant cells, exogenous recombinant IL-1 induces secretion of growth and invasiveness-promoting factors, angiogeni c factors (Apte, R. N., et al. Semin Cancer Biol 2002; 12:277˜290). NIH 3T3 fibroblasts cell lines transfected with secretable IL-1β (mIL-1β and ssIL-1β) were more aggressive than wild-type and mock-transfected tumor cells; ssIL-1β transfectants even exhibited metastatic tumors in the lungs of mice after i.v. inoculation (experimental metastasis). Vascularity patterns were increased in IL-1β tumors. Whereas cells transfected with pIL-1α fail to develop local tumors and activate antitumor effector mechanisms, such as CTLs, NK cells, and high levels of EFN-γ production. The results demonstrate that tumor cell-associated IL-1α potentiates the development of antitumor immune responses, which limit the growth of the malignant cells, whereas IL-1β expression by the tumor cells potentiates invasiveness patterns and induces anergy in the host (Voronov et al., Semin Cancer Biol 2002; 12:277-90). In a murine B16 model, stroma-derived IL-1 promoted melanoma hepatic experimental metastasis, increased liver metastasis following intrasplenic injection of recombinant IL-1β or LPS, a strong IL-1 inducer, while reducted metastases and increased survival rates following treatments with IL-1Ra (Fan et al., Cancer Res 2004; 64:3186-90; Song et al., J immunol 2003; 171:6448-56). Liver metastasis was reduced in IL-1β KO mice and almost completely inhibited in ICE KO mice, in which processing of both IL-1β and IL-18 is inhibited (McKenzie et al., Anticancer Res 1996; 16:437-44). Increased metastasis was mediated through IL-1β up-regulation of expression of VCAM-1 and VLA-4 on HSEs (Fan et al., Cancer Res 2004; 64:3186-90; Song et al., J Immunol 2003; 171:6448-56; McKenzie et al., Anticancer Res 1996; 16:437-44). Using the same IL-1β KO mice, local growth (intrafootpad) of B16 melanoma cells is inhibited and angiogenesis and invasiveness are reduced; in control mice, systemic treatments with IL-1Ra inhibited fibrosarcoma growth (Vidal-Vanaclocha et al., Cancer Res 1994; 54: 2667-2672; Voronov et al., Proc Natl Acad Sci USA. 2003; 100:2645-50). IL-1Ra gene therapy decreases in vivo growth and metastatic potential of a human melanoma xenograft that constitutively secretes IL-1 (Vidal-Vanaclocha et al., Proc Natl Acad Sci USA. 2000; 97:734-9.).

Secreted Form of Interleukin-1

In contrast to other cytokines, IL-1β lacks a secretory signal sequence. Instead, the proform of IL-1β must undergo intracellular, post-translational processing before it can be properly secreted in a bioactive form from a cell. Processing and release is primarily restricted to monocytes and macrophages. When IL-1β has been expressed in cells which lack the intracellular IL-1β processing machinery, IL-1β tends to be retained inside of the cell rather than being secreted. When mature human IL-1β s fused to a signal sequence from the structurally related IL-1 receptor antagonist (IL-1ra), a secreted form of IL-1β is obtained (Wingren et al., Cell Immunol 169:226-237 (1996)). In transgenic mice expressing IL-1β in the specifically in the cells of the spleen and lymph nodes, the IL-1ra signal sequence was sufficient to obtain a secreted form of IL-1β, as detected by IL-1β measurements in sera of the transgenic mice (Bjorkdahl et al., Immunology 96:128-137 (1999)). Those skilled in the art will appreciate that secretory sequences from other secreted proteins can also be utilized in accordance with this invention to obtain a secreted form of IL-1β, a useful secretory sequence is a sequence which causes the protein to which it is fused to be secreted to the extracellular environment.

Tissue-Specific Promoters and Regulatory Sequences

To be expressed, the DNA segment encoding a cytokine must be coupled to a promoter in a functional manner. In the context of this invention, the promoter, including any additional regulatory sequences (i.e., enhancer), is from a gene exclusively or preferentially expressed in a specific tissue. The promoter is operably linked to the DNA segment encoding the cytokine. Within the scope of this invention, other promoters useful for directing tissue-specific transgene expression will be apparent to those skilled in the art.

Stomach

To achieve constitutive cytokine expression in the cells of the stomach, non-limiting examples of promoters directing stomach-specific transgene expression include a H/K-ATPase gene promoter, preferably a H/K-ATPase β subunit gene promoter, or a fragment thereof, or regulatory elements from an Atp4b gene, or a fragment thereof, the keratin 19 (K19) promoter, or a fragment thereof, the FOXa3/HNFγ promoter, or a fragment thereof, trefoil factor (TFF) 1 promoter, or a fragment thereof, TFF2 promoter or a fragment thereof. These promoters and regulatory sequences have been used to direct transgene expression specifically in the stomach of transgenic mice (Li et al., J Biol Chem 270:15777-15788 (1995); Canfield et al., Proc Natl Acad Sci USA 93:2431-2435 (1996); Syder et al., Proc Natl Acad Sci USA 101:4471-4476 (2004)). Trefoil factor (TFF) 1 and TFF2 are proteins that are expressed specifically in the stomach (Terada et al., Biol Phann Bull 24:135-139 (2001)). Thus the TFF1 and TFF2 promoters, or fragments thereof, can be used to direct transgene expression in the stomach. Additional promoters that can be used to achieve transgene expression in the stomach are the keratin 19 (K19) promoter (Brembeck et al., Gastroenterology 120:1720-1728 (2001)), or a fragment thereof, and the FOXa3/HNF3γ promoter, or a fragment thereof.

Lung

To achieve constitutive cytokine expression in cells of the lung, non-limiting examples of promoters directing lung-specific transgene expression include a Clara cell secretory protein (CC10 or CCSP) promoter, or a fragment thereof, or a surfactant protein C (SF-C) promoter, or a fragment thereof. These promoters have been used to express transgenes specifically in cells of the lung (Hicks et al., Toxicology 187:217-228 (2003); Linnoila et al., Exp Lung Res 26:595-615 (2000); Dang et al., Oncogene 22:1988-1997 (2003); Ikeda et al., Am J Physiol 267:L309-L317 (1994)). U.S. Patent Application Publication No. 2004/0244067 and U.S. Pat. Nos. 5,817,911 and 6,566,581 describe the use of the exemplary promoter to generate non-human transgenic mammals with lung-specific expression of various transgenes.

Breast

To achieve constitutive cytokine expression in cells of the breast, non-limiting examples of promoters directing breast-specific transgene expression is a mouse mammary tumor virus (MMTV) promoter, or a fragment therof, or a whey acidic protein promoter or a fragment thereof. These promoters have been used to target transgene expression in the mammary glands of transgenic mice (Wang et al., Nature 369:669-671 (1994); Schoenenberger et al., EMBO J 7:169-175 (1988)). U.S. Pat. No. 6,255,554 describes a whey acidic acid protein and other breast-specific promoters, including casein, lactalbumin and β-lactoglobulin promoters, which can be used to direct transgene expression to the cells of the breast in a transgenic non-human mammal.

Founder lines have been established for a transgenic mouse line carrying an MMTV-IL-1β transgene, designed to overexpress IL-1β in the mammary tissue. The transgene contains the identical recombinant human IL-1β cDNA with the IL-1RA signal peptide provided for by the invention, thus allowing the peptide to be constitutively secreted.

Pancreas

To achieve constitutive cytokine expression in cells of the pancreas, non-limiting examples of promoters directing pancreas-specific transgene expression include a Pdx-1 promoter, or a fragment thereof, an insulin promoter, or a fragment thereof, an elastase promoter, or a fragment thereof, or a phosphoglycerate kinase promoter, or a fragment thereof. These promoters have been used to generate transgenic mice with pancreas-specific transgene expression (Hingorani et al., Cancer Cell 7:469-483 (2005); Elliot and Flavell, Int Immunol 6:1629-1637 (1994); Rajkumar et al., Am J Physiol 270:E565-571 (1996); Nathan et al., Gastroenterology 128:717-727 (2005); Ueno et al., Biochem Pharmacol 60:857-863 (2000)). U.S. Pat. No. 6,716,824 describes the use of a rat insulin promoter to direct transgene expression to the cells of the pancreas in a transgenic non-human mammal.

Founder lines have been established for a transgenic mouse line expressing IL-1β in the pancreas acinar tissue, comprising a rat elastase-IL-1β transgene. The transgene contains the identical recombinant human IL-1β cDNA with the IL-1RA signal peptide provided for by the invention, thus allowing the peptide to be constitutively secreted.

Skin

To achieve constitutive cytokine expression in cells of the skin, non-limiting examples of promoters directing skin-specific transgene expression include keratin promoters such as a K5 promoter, a K6 promoter, or a K14 promoter, or a fragment thereof. These promoters have been used to generate transgenic mice with skin-specific transgene expression (Cataisson et al., J Immunol 171:2703-2713 (2003); Shibaki et al., J Invest Dermatol 123:109-115 (2004); Feith et al., Cancer Res 61:6073-6081 (2001) Vassar et al., Proc Natl Acad Sci USA 86:1563-1567 (1989)). Additionally, U.S. Pat. No. 5,811,634 describes the use of promoter/regulatory sequences of a K1 promoter, a K5 promoter, a K6 promoter and a K10 promoter to direct transgene expression to the cells of the skin in a transgenic non-human mammal.

Esophagus

To achieve constitutive cytokine expression in cells of the esophagus, a non-limiting example of a promoter directing esophagus-specific transgene expression is an Epstein-Barr virus (EBV) ED-L2 promoter, or a fragment thereof. The EBV ED-L2 promoter has been used to express a transgene in the cells of the esophagus of transgenic mice (Opitz et al., J Clin Invest 110:61-769 (2002); Fong et al., Cancer Res 63:4244-4252 (2003)).

Liver

To achieve constitutive cytokine expression in cells of the liver, non-limiting examples of promoters directing liver-specific transgene expression are a major urinary protein (MUP) promoter, or a fragment thereof, an albumin promoter, or a fragment thereof, a transthyretin promoter, or a fragment thereof, an apoE promoter, or a fragment thereof, or a phenylalanine hydroxylase promoter, or a fragment thereof. These promoters have been used to generate transgenic mice with transgene expression directed to the cells of the liver (Kawamure et al., Hepatology 25:1014-1021 (1997); Kuklin et al., Mol Cancer 3:17-27 (2004); Nicolas et al., Proc Natl Acad Sci USA 99:4596-4601 (2002); FEBS Lett 555:528-532 (2003); Jackerott et al., Diabetologia 45:1292-1297 (2002)).

Colon

To achieve constitutive cytokine expression in cells of the colon, non-limiting examples of promoters directing colon-specific transgene expression are a villin promoter, or a fragment thereof, and a fatty acid binding protein promoter (FABP), or a fragment thereof. These promoters have been used to generate transgenic mice with colon-specific transgene expression (Pinto et al., J Biol Chem 274:6476-6482 (1999); Janssen et al., Gastroenterology 123:492-504 (2002); Sweetser et al., Proc Natl Acad Sci 85:9611-9615 (1988); Cobb et al., Cancer 100″1311-1323 (2004)). U.S. Application Publication No. US 2003/0177516 describes the use of the intestinal FABP promoter region to direct transgene expression to the cells of the gut in a transgenic bird.

Prostate

To achieve constitutive cytokine expression in cells of the prostate, non-limiting examples of promoters directing prostate-specific transgene expression are a cryptdin-2 promoter, or a fragment thereof, a prostate-specific antigen (PSA) promoter, or a fragment thereof, a C(3)1 promoter, or a fragment thereof, prostate secretory protein of 94 amino acids (PSP94) promoter, or a fragment thereof, and a probasin promoter, or a fragment thereof. These promoters have been used to generate transgenic mice with prostate-specific transgene expression (Garabedian et al., Proc Natl Acad Sci USA 95:15382-15387 (1998); Cleutjens et al., Mol Endocrinol 11:1256-1265 (1997); Zhang et al., Prostate 443:278-285 (2000); Gabril et al., Gene Ther 9:1589-1599 (2002); Masumori et al., Cancer Res 61:2239-2249 (2001)). U.S. Pat. No. 6,136,792 describes the use of promoter/regulatory sequences of the PSA promoter to direct transgene expression to the cells of the prostate in a transgenic non-human mammal. Additionally, U.S. Pat. Nos. 5,952,488 and 5,907,078 describe the use of the probasin promoter to drive expression of a transgene specifically in the prostate of transgenic non-human mammals.

Ovary

To achieve constitutive cytokine expression in cells of the ovaries, a non-limiting example of a promoter directing ovary-specific transgene expression is the ovarian-specific promoter (OSP-1). This promoter has been used to express a transgene in the cells of the ovaries of transgenic mice (Garson et al., J Soc Gynecol Investig 10:244-250 (2003)).

Kidney

To achieve constitutive cytokine expression in cells of the kidneys, non-limiting examples of promoters directing kidney-specific transgene expression are the uromodulin promoter, or a fragment thereof, Tamm-Horsfall protein (THP) promoter, or a fragment thereof, and the type 1 gamma-glutamyl transpeptidase promoter or a fragment thereof. These promoters have been used to generate transgenic mice with kidney-specific transgene expression (Huang et al., BMC Biotechnol 5:9 (2005); Zhu et al., Am J Physiol Renal Physiol 282:F608-F617 (2002); Terzi et al., J Clin Invest 106:225-234 (2000)). U.S. Pat. No. 6,888,047 describes the use of the uromodulin promoter to direct transgene expression to the cells of the kidneys in a transgenic non-human mammal.

Bladder

To achieve constitutive cytokine expression in cells of the bladder, non-limiting examples of promoters directing bladder- or urothelium-specific transgene expression are the promoters, or fragments thereof, directing expression of the uroplakin genes. The uroplakin II promoter has been used to engineer transgenic mice with transgene expression specifically in urothelium of the bladder (Cheng Cancer Res 62: 4157-4163 (2002); Lin et al., Proc Natl Acad Sci USA 92:679-683 (1995)). U.S. Pat. Nos. 5,824,453 and 6,001,646 describe the use of the uroplakin II gene promoter to produce transgenic animals expressing a transgene specifically in the cells of the bladder urothelium. Furthermore, U.S. Pat. No. 6,339,183 describes transgenic animals in which the urothelium-specific expression of a transgene is directed by a uroplakin Ia promoter, a uroplakin mi promoter or a urohingin promoter.

Uterus

To achieve constitutive cytokine expression in cells of the uterus, a non-limiting example of a promoter directing uterus-specific transgene expression is the uteroglobin promoter, or fragment thereof. This promoter has been used to engineer transgenic mice with transgene expression specifically in the uterus (Gomez Lahoz et al., Gene 117:255-258 (1992); Sandmoller et al., 9:2805-2815 (1994)).

Screening Methods

Using a transgenic mammal provided for by the present invention, compounds are screened for their ability to treat cancer, or cause, prevent or delay the onset of cancer. Accordingly, the present invention provides for a method for identifying whether a test compound is capable of treating cancer, the method comprising (a) administering an effective amount of a test compound to a transgenic non-human mammal encompassed by the present invention; (b) measuring progression of cancer in the transgenic non-human mammal of (a); and (c) comparing the progression of cancer measured in (b) to progression of cancer measured in a sibling of the transgenic non-human mammal, wherein the sibling was not administered the test compound, and wherein a decrease in progression of cancer in the non-human mammal of (a) compared to (b) indicates that the test compound is capable of treating cancer. In one embodiment, the transgenic non-human mammal has cancer. In another embodiment, a decrease comprises an arrest, delay or reversal in progression of cancer.

In a further embodiment, the measuring comprises a histological assessment, an assessment of alterations in the mammal's weight and activity, non-invasive imaging, an assessment of serum biomarkers, or any combination thereof. The diagnostic utility of the serum biomarkers IL-6 and C-reactive protein (CRP) is well-documented (Chung and Chang, J Surg Oncol, 83:222-226 (2003); Alexandrakis et al., Am J Hematol 72:229-233 (2003); Mahmoud and Rivera, Curr Oncol Rep, 4:250-255 (2002); Deichmann et al., J Exp Clin Cancer Res 19:301-307 (2000); Lehrnbecher et al., Clin Infect Dis 29:414-419 (1999); Costes et al., J Clin Pathol 50:835-840 (1997)). Exemplary methods for non-invasive imaging of cancer include micro-CT, PET and SPECT imaging and bioluminescence imaging (Choy et al., Mol Imaging 2:303-312 (2003); DeClerck et al., Neoplasia 6:374-379 (2004); Cancer Res 61:110-117 (2000)).

In one embodiment, the cancer comprises a breast cancer, a colon cancer, a pancreatic cancer, a lung cancer, an ovarian cancer, a cervical cancer, a uterine cancer, a bone cancer, a stomach cancer, a gastric cancer, a testicular cancer, a prostate cancer, a skin cancer, an esophageal cancer, a liver cancer, a kidney cancer, a bladder cancer, a lymphoma, or any combination thereof. Specifically, the lymphoma may comprise a B-cell lymphoma.

This invention additionally provides for a method for identifying whether a test compound is capable of preventing or delaying the development of cancer, the method comprising (a) administering an effective amount of a test compound to a transgenic non-human mammal encompassed by this invention, wherein the transgenic non-human mammal does not have cancer; (b) measuring development of cancer in the transgenic non-human mammal of (a); and (c) comparing the development of cancer measured in (b) to development of cancer measured in a sibling of the transgenic non-human mammal, wherein the sibling was not administered the test compound, and wherein inhibition of or a delay in the development of cancer in the non-human mammal of (a) compared to (b) indicates that the test compound is capable of preventing or delaying the development of cancer. In one embodiment, the cancer comprises a breast cancer, a colon cancer, a pancreatic cancer, a lung cancer, an ovarian cancer, a cervical cancer, a uterine cancer, a bone cancer, a stomach cancer, a gastric cancer, a testicular cancer, a prostate cancer, a skin cancer, an esophageal cancer, a liver cancer, a kidney cancer, a bladder cancer, a lymphoma, or any combination thereof.

A method for identifying whether a test compound is a carcinogen, the method comprising (a) administering to a transgenic non-human mammal of this invention or exposing a transgenic non-human mammal of this invention to an effective amount of a test compound, wherein the transgenic non-human mammal does not have cancer; (b) measuring development of cancer in the transgenic non-human mammal of (a); and (c) comparing the development of cancer measured in (b) to development of cancer measured in a sibling of the transgenic non-human mammal, wherein the sibling was not administered the test compound or exposed to the test compound, and wherein earlier development of cancer in the non-human mammal of (a) compared to (b) indicates that the test compound is a carcinogen. In one embodiment, the cancer comprises a breast cancer, a colon cancer, a pancreatic cancer, a lung cancer, an ovarian cancer, a cervical cancer, a uterine cancer, a bone cancer, a stomach cancer, a gastric cancer, a testicular cancer, a prostate cancer, a skin cancer, an esophageal cancer, a liver cancer, a kidney cancer, a bladder cancer, a lymphoma, or any combination thereof.

Cell Culture

The transgenic non-human mammals of the present invention can be used as a source of cells for cell culture. Tissues of the transgenic mammals are analyzed for the expression of the cytokine encoded for by the transgene. Cells of tissues expressing the transgene can be cultured using standard cell and tissue culture techniques. Provided for by this invention is a cell from a non-human transgenic mammal encompassed by this invention.

The following examples illustrate the present invention, and are set forth to aid in the understanding of the invention, and should not be construed to limit in any way the scope of the invention as defined in the claims which follow thereafter.

### EXAMPLES

### Example 1

### Overexpression of Interleukin-1b Induced Gastric Inflammation and Carcinogenesis in Transgenic Mice

Interleukin-1β (IL-1β) is a multifunctional, proinflammatory cytokine that has profound effects on immunity and gastric physiology (for example, inhibition of gastric acid secretion). IL-1β polymorphisms have been associated with an increased risk of gastric cancer, although direct evidence for a pathogenic role in cancer has been lacking. The invention provides for a novel human IL-1β (hIL-1β) transgenic mouse with targeted expression of a mature secreted type of hIL-1β cDNA to the stomach using the mouse H/K-ATPaseβ promoter. The transgenic mouse provided by the invention can be used to study the effects and mechanism of human IL-1β overexpression in gastric inflammation and carcinogenesis.

As described in this Example, the hlL-1β transgenic mice provided by the invention expressed high levels of human IL-1β in the gastric mucosa but not in the other tissues. IL-1β transgenic mice exhibited hypochlorhydria and hypergastrinemia, and mice greater than 1 year of age developed spontaneous foveolar hyperplasia, acute and chronic inflammation, metaplasia and dysplasia. More importantly, male IL-1β, transgenic mice progressed by 12-16 months of age to spontaneous gastric cancer. Notably, IL-1β transgenic mice exhibited enlarged spleens and a significant increase in the number of activated T cells, neutrophils and macrophages and a significant decrease in B cells in the stomach and spleen tissues. The angiognenesis and the expression of TFF2, Cox-2, MMP-9 and VEGF in the stomach were significantly increased in IL-1β transgenic mice. The expression levels of TNF-α and IL-6 were significantly upregulated in the stomach and sera of IL-1β transgenic mice. This Example demonstrates for the first time that overexpression of IL-1β even in the absence of Helicobacter pylori infection, can induce spontaneous gastric inflammation and dysplasia (carcinoma), possibly through inhibition of gastric acid, upregulation of cytokines and dysregulation of immunity. IL-1β plays an important role in carcinogenesis, and represents a possible target for prevention and treatment of stomach cancer.

The relationship between chronic inflammation and cancer has long been recognized. While it was Galen who first noted the relationship two millennia ago, Rudolf Virchow in 1963 demonstrated the presence of leukocytes in malignant tissues and hypothesized that the origin of cancer was at sites of chronic inflammation (42). Cancer in recent years has been viewed as “the wound that will not heal”, and many solid malignancies appear to be initiated by tissue injury or chronic inflammation, which can often be linked to known bacterial, viral or parasitic infections. Population-based studies show that susceptibility to cancer increases when tissues are chronically inflamed, and long-term use of NSAIDs reduces the risk of many cancers.

The microenvironment around tumors typically contains a variety of cells of the innate immune system, and a number of studies indicate that macrophages promote the development, growth, angiogenesis and invasiveness of cancer cells. Recent reports have demonstrated that the transcription factor NF-κB is a key player linking inflammation and cancer, primarily through its role in macrophages leading to the activation of a number of downstream targets, including cytokines, growth factors, adhesion molecules, metalloproteinases and other enzymes. Conditional deletion of EKKβ in myeloid cells leads to a reduction in colorectal and hepatic cancers in mice (43,44). In addition, numerous studies in patients have shown a strong correlation between macrophage infiltration and tumor vascularity and progression, including studies in gastric cancer (45). However, the critical downstream targets of NF-κB within myeloid cells have not been clearly defined.

Interleukin-1β (IL-1β) is proinflammatory pleiotropic cytokine that affects mainly hematopoiesis, inflammation, immunity and tumorigenicty (1-2). The properties of IL-1 stem from its ability to induce the synthesis of cytokines, chemokines, proinflammatory molecules, and the expression of adhesion molecules (1). The IL-1 gene family consists of two major agonistic molecules, namely IL-1α and IL-1β, and one antagonistic cytokine, the EL-1R antagonist (IL-1RA) (3). IL-IRA binds to the IL-1 receptor type I without transmitting an activation signal and, thus, represents a physiological inhibitor of IL-1 activity. IL-1α and IL-1β bind to the same receptors, and there are no significant differences in the spectrum of activities induced by recombinant IL-1α or IL-1β by using in vitro and in vivo assays, including injection into humans (1).

Many studies have showed that IL-1β is involved in angiogenesis, tumorigenesis and invasion (4). IL-1β markedly induced angiogenesis in vitro and in vivo by upregulation of expression of various prostanoids (4-5). In microenvironmental stroma cells and in malignant cells, exogenous recombinant IL-1 induces secretion of growth and invasiveness-promoting factors, angiogenic factors (1). Recently, reports showed that NIH 3T3 fibroblasts cell lines transfected with secretable IL-1β (mIL-1β and ssIL-1β) were more aggressive than wild-type and mock-transfected tumor cells; ssIL-1β transfectants even exhibited metastatic tumors in the lungs of mice after i.v. inoculation (experimental metastasis). Vascularity patterns were increased In IL-1β tumors. Whereas cells transfected with pIL-1α fail to develop local tumors and activate antitumor effector mechanisms, such as CTLs, NK cells, and high levels of IFN-g production. The results demonstrate that tumor cell-associated IL-1α potentiates the development of antitumor immune responses, which limit the growth of the malignant cells, whereas IL-1β expression by the tumor cells potentiates invasiveness patterns and induces anergy in the host (6). In a murine B16 model, stroma-derived IL-1 promoted melanoma hepatic experimental metastasis, increased liver metastasis following intrasplenic injection of recombinant IL-1β or LPS, a strong IL-1 inducer, while reduced metastases and increased survival rates following treatments with IL-1RA (7-8). Furthermore, liver metastasis was reduced in IL-1β KO mice and almost completely inhibited in ICE KO mice, in which processing of both IL-1β and IL-18 is inhibited (9). Increased metastasis was mediated through IL-1β up-regulation of expression of VCAM-1 and VLA-4 on HSEs (10-11). Using the same IL-1β KO mice, local growth (intrafootpad) of B16 melanoma cells is inhibited and angiogenesis and invasiveness are reduced; in control mice, systemic treatments with IL-1RA inhibited fibrosarcoma growth (9). IL-1RA gene therapy decreases in vivo growth and metastatic potential of a human melanoma xenograft that constitutively secretes IL-1(12). A continuous delivery of a low, but steady-state level of the naturally IL-1RA from microencapsulated genetically engineered cells, reduced inflammatory responses and inhibited tumor development in mice, phenomena that are induced by IL-1. Release of 25 ng per day of the IL-1RA inhibited tumor development and reduced angiogenesis. These data strongly suggest that IL-1β plays an essential role in cancer development.

The pro-inflammatory cytokine IL-1β has a strong link to NF-κB, in that it both activates and is activated in macrophages by NF-κB, thus creating a positive regulatory loop that can amplify and sustain an inflammatory response. IL-1β is not constitutively expressed in healthy individuals, but is strongly induced by infection, hypoxia and many other types of stresses and disease states. In fact, there appears to be a very narrow margin between physiologic and toxic levels of IL-1β, and consequently, both the synthesis of and responses to IL-1β are tightly controlled. The production of IL-1β is regulated at both the transcriptional and translational levels, and in addition IL-1β undergoes post-translational processing. IL-1β is produced as a pro-form without a signal sequence, and following synthesis, pro-IL-1β remains primarily cytosolic until it is cleaved by caspase-1/ICE and transported out of the cell. In addition, the receptor for IL-1β includes a complex of IL-IR1/IL-R1-AcP as well as a decoy receptor (IL-1RII) and a natural antagonist, IL-1RA. IL-1RA binds to the IL-1 receptor typeI without transmitting an activation signal and, thus, represents a physiological inhibitor of IL-1 activity. Indeed, there is evidence that the balance of IL-1β and IL-1RA may influence the inflammatory response and disease outcome.

Gastric cancer is a result of interaction between genetic factors of the host together with diet and other factors in the environment. Epidemiological studies on Northern Chinese and American Japanese in Hawaii lend strong support to the effects of lack of fresh fruit and vegetable, smoking, and consumption of salty food in the development of gastric cancer. While IL-1β has been associated with a number of solid malignancies, it has been linked particularly strongly to gastric cancer. While gastric cancer has been declining in the U.S., it remains the second most common cause of cancer-related mortality in the world. It is clear that Helicobacter pylori (H. pylori) plays a pivotal role in triggering chronic inflammation of the stomach, leading to stepwise development of malignancy (13), but only 1% of infected individuals will after many years of Helicobacter infection develop gastric cancer, a multistep process that involves histopathologic progression from chronic gastritis to gastric atrophy, intestinal metaplasia, dysplasia and finally gastric cancer (46). The variable response to this common pathogen appears to be governed by a genetic predisposition to high levels of expression of pro-inflammatory cytokines.

Recently, the IL-1β gene has been proposed as a key factor in determining the pattern of gastritis and risk of malignant transformation (14-15). The biological effects of IL-1 as a potent proinflammatorycytokine and inhibitor of acid suppression fits with Correa's model. IL-1β has been shown to be induced by H. pylori infection (16) and is also known to be a strong inhibitor of acid secretion in the stomach (17-18), as well as a strong proinflammatory cytokine. The IL-1 gene cluster (IL-1β encoding IL-1β and IL-1 RN encoding the IL-1 receptor antagonist) has a number of functionally relevant polymorphisms that could be correlated with high or low IL-1β production. Carriers of the IL-1B polymorphisms (IL-1B-511IT and IL-1B-31C) linked to enhanced IL-1β production showed an increased risk for gastric cancer (19-21). These IL-1B-511T and IL-1B-31C alleles enhance IL-1β production and the circulating levels of the cytokine in humans (19-20). Additional studies have shown an association between IL-1β polymorphisms and an increased risk of other solid malignancies (47-51). The association between IL-1 genotypes and gastric cancer was confined to the intestinal type but not diffuse type (19-22). These data showed that IL-1β polymorphisms are associated with an increased risk of gastric cancer development. However, direct evidence in which IL-1β induces stomach carcinogenesis remains to be obtained.

The invention provides a human IL-1β (hIL-1β) transgenic mouse targeting expression of human IL-1β to the stomach. This Example shows that IL-1β transgenic mice develop spontaneous inflammation, dysplasia and carcinoma in stomach. The mechanisms may involve inhibition of gastric acids, proinflammatory cytokines, and dysregulation of immunity. IL-1β plays a pivotal role in development of gastric carcinogenesis, and represents a possible therapeutic target for prevention and treatment of stomach cancer.

Methods

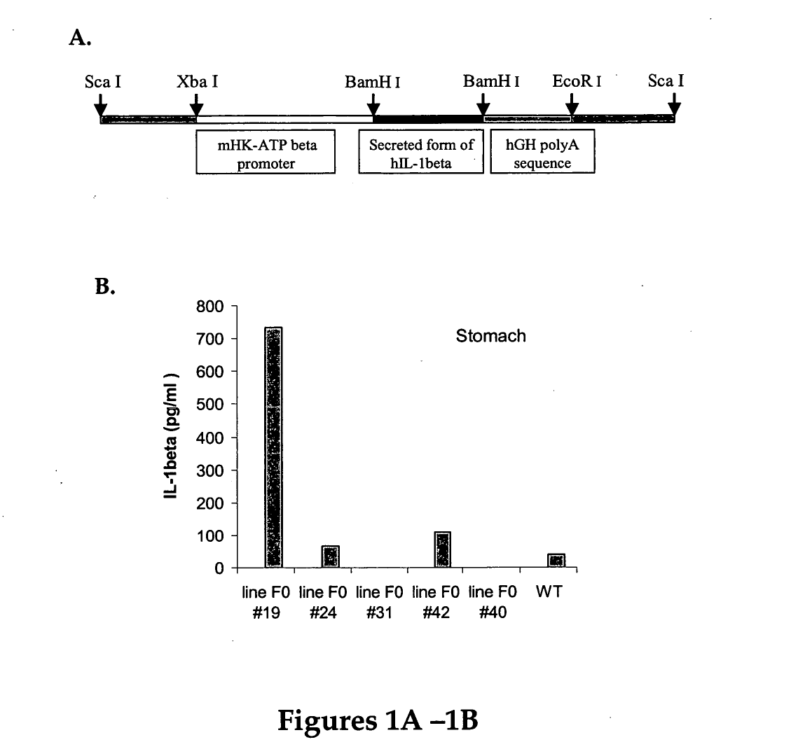

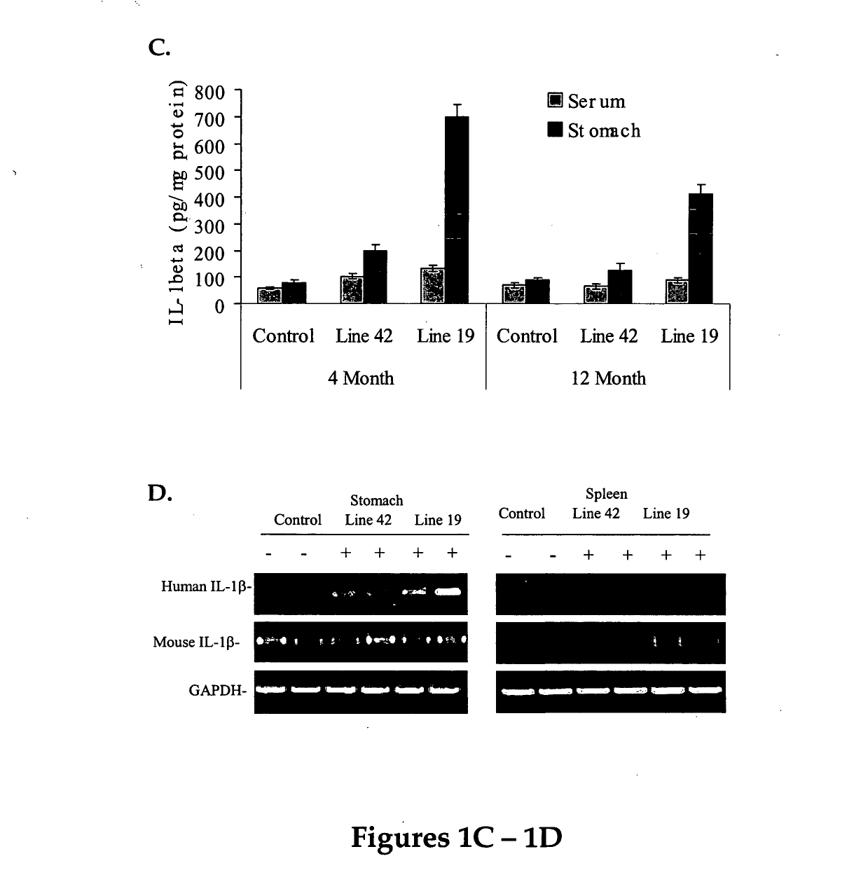

Generation of HK-ATPase hIL1-beta Transgenic Mice. Mouse HK-ATIIασεβ subunit promoter 1,060 bp fragment was used (23). The 550 bp cDNA construct was used (24). It consisted of a secreted form of the mature human IL-1β cDNA fused with the signal sequence derived from the structurally related human IL1-RA (FIG. 1A). These fragments were subcloned together with a human growth hormone polyadenylation sequence into pBluescript vector.

The transgene construct was linearized by ScaI digestion and used for pronuclear injection of C57BL/6×SJL F2 hybrid zygotes. Injected zygotes were implanted into pseudopregnant recipients, and a total of 49 potential F0 founders were produced. Through tail DNA PCR and southern blot analysis, a total of 8 positive founders were obtained and backcrossed to C577BL/6J mice. The transgenic mice with high expression of hIL-1β in the stomach tissues were screened using ELISA from 8 positive F1 pups. A line of transgenic mice was obtained with high expression of hIL-1β in the stomach tissues (line 19) and two lines with low expression of hIL-1β (line 42 and line 24). Line 19 and line 42 mice were chosen to mate with C57BL/6 mice for further study.

Quantitative and semiquantitative polymerase chain reaction. Total RNA was isolated from the stomachs of IL-1β transgenic mice and C57BL/6J control mice. Reverse transcription was performed using SuperScript III First-Strand Synthesis System (Invitrogen) according to the manufacturer's instructions. The semiquantitative PCR reactions were performed with a GeneAmp PCR System 9700 (Applied Biosystems) using PCT Core Kit according to the manufacturer's instructions. The cDNA was standardized to give similar amounts of glyceraldehyde triphosphate dehydrogenase (GAPDH) amplification per microliter of diluted cDNA. One microliter of cDNA was amplified with 5 pmol of each sense and antisense primer, 10 pmol of deoxynucleoside triphosphates, 1 U Taq DNA polymerase (Qiagen) in 10 mmol/L Tris/HCl (pH 8.3) containing 50 mmol/L KCl, 4.0 mmol/L MgCl₂, and Q buffer at the recommended concentration in a total volume of 20 μL. After an initial denaturation (2 minutes at 94° C.), the samples were subjected to 35-45 cycles of 94° C. for 30 seconds, 60° C. for 30 seconds, 72° C. for 20 seconds, and 72° C. for 5 minutes. Reactions were performed for GAPDH, EL-1β, IFN-r, TNF-α, IL-4, IL-6 and IL-10. The resulting PCR products were analyzed by agarose gel electrophoresis. Quantitative real-time PCR was performed with a 3-step method using the Bio-Rad iCycler iQ real-time PCR detection system (Bio-Rad Laboratories). Each reaction was carried out in a 50 μL mixture consisting of iQ SYBR Green Supermix (Bio-Rad Laboratories) or QuantiTect SYBR Green PCR (Qiagen), additional MgCl₂ for a final MgCl₂ concentration of 4.0 mmol/L, 0.2 μmol/L of each primer, and 1 μL of template cDNA. The sense and the antisense primers were designed to cross exon-intron boundaries to avoid amplification from contaminating DNA. The product sizes and the sequences of the primers are shown in Table 1.

TABLE 1
The primers for PCR and Real-time PCR

| | | |
|---|---|---|
| Genes | Sequences | Size of Product |

| | | | | |
|---|---|---|---|---|
| hIL-1β | Forward: | 5′-tgc gaa tct ccg acc acc act aca g-3′ | (SEQ ID NO:1) | 295 bp |
| | Reverse: | 5′-tgg agg tgg aga gct ttc agt tca tat g-3′ | (SEQ ID NO:2) | |
| mIFN-γ | Forward: | 5′-cat ggc tgt ttc tgg ctg tta ctg-3′ | (SEQ ID NO:3) | 226 bp |
| | Reverse: | 5′-gtt gct gat ggc ctg att gtc ttt-3′ | (SEQ ID NO:4) | |
| mTNF-α | Forward: | 5′-tgg ccc aga ccc tca cac tca g-3′ | (SEQ ID NO:5) | 180 bp |
| | Reverse: | 5′-acc cat cgg ctg gca cca ct-3′ | (SEQ ID NO:6) | |
| mIL-1β | Forward: | 5′-gga gaa cca agc aac gac aaa ata-3′ | (SEQ ID NO:7) | 211 bp |
| | Reverse: | 5′-tgg gga act ctg cag act caa ac-3′ | (SEQ ID NO:8) | |
| mIL- | Forward: | 5′-ctg cct gcc ccc aca gaa ga-3′ | (SEQ ID NO:9) | 208 bp |
| 12α | Reverse: | 5′-gcg cag agt ctc gcc att atg a-3′ | (SEQ ID NO:10) | |
| mIL-6 | Forward: | 5′-gtt ttc tgc aag tgc atc atc g-3′ | (SEQ ID NO:11) | 236 bp |
| | Reverse: | 5′-gtt ttc tgc aag tgc atc atc g-3′ | (SEQ ID NO:12) | |
| mIL-4 | Forward: | 5′-atc ggc att ttg aac gag gtc a-3′ | (SEQ ID NO:13) | 221 bp |
| | Reverse: | 5′-cat cga aaa gcc cga aag agt ct-3′ | (SEQ ID NO:14) | |
| mIL-10 | Forward: | 5′-cct aga gct gcg gac tgc ctt ca-3′ | (SEQ ID NO:15) | 247 bp |
| | Reverse: | 5′-cag ccg cat cct gag ggt ctt c-3′ | (SEQ ID NO:16) | |
| mGAPDH | Forward: | 5′ gga gaa acc tgc caa gta tg-3′ | (SEQ ID NO:17) | 256 bp |
| | Reverse: | 5′-tgg gag ttg ctg ttg aag tc-3′ | (SEQ ID NO:18) | |

(Note: h: human, m: mouse)

All primer pairs were optimized to amplify only a single band, with amplification curves in real-time PCR consistent with efficient amplification under the reaction conditions. The PCR conditions were as follows: 95° C. for 3 minutes, followed by 50 cycles of 95° C. for 30 seconds, 60° C. for 30 seconds, and 72° C. for 30 seconds.

Determination of Gastric Acid Secretion and Gastrin. Five to ten IL-1β and control mice at ages of 12 months were selected for determination of acid secretion via the pyloric ligation technique. Gastric juice was collected and measured with a pH meter (AR 25, Fisher Scientific, Houston, Tex.) by 0.01 N NaOH titration, and results were expressed as micro-equivalents. Human amidated gastrin-17 levels were measured using the L6 antibody, and total amidated gastrins (human and mouse) were measured using the L2 antibody in RIAs as described previously (Wang et al., Gasteroenterology 118:36-47 (2000)).

Measurement of cytokine levels by ELISA. The levels of hEL-1β, TNF-α and IL-6 in serum or stomach tissues of the transgenic mice were determined by ELISA (BD Company) according to the manufacturer's instructions. The standard recombinant cytokines, hIL-1β, mouse TNF-α and IL-6 (BD Company) were used. Absorbance was measured at 450 nm by a Multiscan MC reader, and the samples were analyzed by DELTA SOFT II software (BioMetallics, Inc.). Measurement of cytokine content was performed in the linear part of the standard curve.

Histopathological analysis and Immunohistochemistry. The stomach, spleen and other tissues from transgenic and control mice were fixed in 10% formalin before embedded in paraffin. Sections (5 μm) of these tissues were stained with eosin/haematoxylin (H&E) for pathological analysis. Indices of injury in the gastric cardia/corpus and antrum were scored on an ordinal scale from 0 to 4 in increments of 0.5 by a single pathologist blinded to treatment groups (25,26). The tissue examined consisted of a section of gastric mucosa taken from the lesser curvature of the stomach beginning at the gastroesophagealjunction and ending at the gastroduodenal junction. The glandular mucosa of the fundic and antro-pyloric regions were given separate histological scores from 0 to 4 (normal, minimal, mild, moderate, and marked) for inflammatory criteria including mucosal lymphoplasmacytic infiltration, mucosal granulocyte infiltration, and submucosal lymphoid follicle development. Inflammation was distinguished histologically into chronic (lymphohistiocytic) and active (granulocytic) components. The contributions of each were graded on an ascending scale, from 0 to 4, based on the intensity, distribution and confluence of inflammatory infiltrates. Assessment of mucosal alterations was based on atrophy of glandular cells and epithelial hyperplasia; scores of 0 to 4 were assigned based on histologic estimation of the percentage of altered mucosa: 0, no substantial alteration; 1, less than 5%; 2, 25-50%; 3, 50-75%; and 4, more than 75%. Mucus metaplasia observed on H&E sections was verified by examination of changes in the epithelial cell-staining patterns on Alcianblue. Dysplasia was graded according to previously described criteria (57,58). The presence or absence of invasion of atypical tissue into the submucosal stroma and associated endothelium-lined structures were noted (25,26).

Immunohistochemistry. Sections were blocked for 15 min in avidin, washed in PBS and then blocked for 15 min in biotin (Avidin/Biotin blocking kit; Vector Labs, Burlingame, Calif.; 4 droplets/ml diluted in PBS) before staining. The following primary antibodies were used: H⁺K⁺-ATPaseβ subunit to parietal cells (1:2,000, mouse antihog, Affinity Bioreagents), antibody Ki-67 (DAKO), TFF2, NF-κB p65 antibody (Santa Cruz), CD34 (Abcam), Cox-2 (Cayman), VEGF (Abeam), MMP (Santa Cruz) and control rat IgG2a. Primary antibodies were incubated at 4° C. overnight in humidified chamber. Staining with primary antibodies was followed by further incubation with biotinylated goat antirat IgG (Jackson Immunoresearch Laboratories Inc.) at a 1:400 dilution, for 30 min. The sections were then incubated for 30 min with ABC Elite horseradish peroxidase (Vector Labs). The antigen-antibody complexes were made visible using diaminobenzidine (DAB) (Vector Labs) for 5 min. The slides were counterstained in methyl green and mounted in DPX medium (DAKO or Kebo Lab AB). The following primary antibodies were used: H⁺K⁺-ATPasep subunit to parietal cells (1:2,000, mouse antihog, Affinity Bioreagents), against chromogranin A to stain enterochromaffin-like (ECL) cells (working dilution 1:500, rabbit antiporcine, Immunostar), as well as antibody Ki-67, TFF2, NF-κb, CK-7 and control rat IgG2a. Primary antibodies were incubated at 4° C. overnight in humidified chamber.

Microvessel density (MVD) was evaluated according to method described previously. MVD was the average of the vessel counts obtained in the three sections. Areas of the highest neovascularization were chosen, and microvessel counting was performed at 200× magnification in three chosen fields. Any immunoreactive endothelial cell or endothelial cell cluster that had been distinctly separated from adjacent microvessels was considered a single countable vessel. The results regarding angiogenesis in each tumor were expressed as the absolute number of vessels/0.74 mm² (200× field). In all assays, matched isotype control antibodies were used and found to be nonreactive in all cases.

Western Blot analysis. Gastric tissues from IL-1β mice and control mice were homogenized in 0.3-0.5 ml ice-cold lysis buffer containing 8 mmol/L Na₂HPO₄, 3 mmol/L NaH₂PO₄ and EDTA-free protease inhibitor (Roche Applied Science), and sonicated. Protein concentration was determined using a kit (Bio-Rad Laboratories) and 50 μg of protein was loaded onto 12% SDS-polyacrylamide separating gel. After electrophoresis, proteins were transferred to nitrocellulose membrane. The blots were incubated with primary antibody anti-VEGF, Cox-2 and MMP-9 antibody, respectively. These antibodies were visualized with an enhanced chemiluminescence system (Amersham).

Bone marrow transplantation, and Helicobacter felis infection and IL-RA treatment. Bone marrow was isolated from the femurs and tibias of 6-8 week old C57BL/6-TgN [ACThEGFP] (GFP) mice. Total bone marrow was washed, triturated using a 20 gauge needle and passed through a 40 sum nylon mesh cell strainer (Becton Dickson) to produce a single cell suspension in PBS. Recipient IL-β mice and C57BL/6J mice were irradiated with 900 rads from a cesium 137-gamma cell irradiator, reconstituted with 3×10⁶ donor marrow cells via a single tail vein injection, and used for experiments after 2 weeks of recovery. The overall level of engraftment was 60-80% as assessed by analysis of β-galactosidase or GFP in peripheral leukocytes. Non-transplanted IL-β mice and C57BL/6 mice served as controls for all injury models. Helicobacter felis (strain 49179) was obtained from the American Type Cell Culture (Rockville, Md.), grown as recommended and bacteria enumerated as previously described (Houghton 2004). Two weeks after transplantation, mice were infected by oral gavage with 1×10⁷ colony forming units every other day for 3 days. In the same day, mice received the treatment of IL-1 receptor antagonist (hIL-1RA) by continuously infused using an osmotic minipump (Alzet 2 mL2, Alza Corporation, Palo Alto, Calif.) implanted subcutaneously (i.p.), with the intention of delivery a dose of 100 mg/kg/day per mouse for 30 days (20 mg per mouse). Control groups will be given saline. Eight weeks after infection, mice were euthanized, and bone marrow, stomach, spleen and blood were collected for FACS and immunofluorecence assays. All animal work was performed at Columbia University under approval of the Columbia University Institutional Animal Care and Use Committee.

Single cell preparation and FACS analysis. Eight weeks after H. felis infection, retro-orbital blood was collected immediately before sacrifice. Total nuclear cell in peripheral blood were isolated by erythrocyte lysis with ammonium chloride solution (PharM Lyse). For single cell suspension preparation from stomach tissues, stomachs were removed, opened and extensively washed. The fundic mucosa was gently scraped free from the serosa, minced and digested for 2 hours in 1 mM EDTA, 2% BSA and 0.1% pronase in PBS at 37° C. and filtered through a 40 pm nylon mesh strainer. The cells were resuspended in Dulbecco's PBS (D-PBS). For single splenic cell suspension, spleens were islolated from IL-1β mice and control mice. The spleens were disaggregated in cold Hanks balanced salt solution, clumps were allowed to settle out, and the supernatant was sedimented at 1,000 rpm in an EEC PR-600 centrifuge for 10 min. Erythrocytes were removed by hypotonic lysis, and filtered through a 40 μm nylon mesh strainer. The splenocytes were resuspended in D-PBS. All single cell suspensions were stained with FITC-labeled anti-CD34, PE-labeled anti-SCN as well as Cy5-labeled anti-CD3 and anti-B220 antibodies (Pharmingen) and analyzed using a FACScan™ flow cytometer.

Immunofluorescence. Two pieces of stomach and spleen were removed and snap-frozen by liquid nitrogen for standard H&E and immunofluorescence staining. Five micrometers frozen sections were stained with FITC-labeled CD3, CD4, CD8, CD19, CD11b and anti-F4/80 antibody (Santa Cruz) with anti-FITC-conjugated rabbit antibody (Vector), as well as HSCs marker anti-PE-labeled CD133 antibody, anti-Cy5-labeled CD34, PE-labeled anti-SCN (Pharmingen). Specimens were observed with Olympus Fluoview Confocal Microscope and images were analyzed with Adobe Photoshop soft program. Nine fields from two tree samples were randomly selected for statistical evaluation.

Results

Human IL-1β was specifically overexpressed in gastric mucosa of IL-1β transgenic mice. Since IL-1β genetic polymorphisms are associated with risk for gastric cancer, studies were designed to investigate the direct effect of expression of IL-1β on gastric carcinogenesis in vivo buy using a transgenic mouse model. In order to target the expression of hIL-1β to the stomach tissues, the hIL-1β expression vector was constructed (FIG. 1A). This construct was microinjected into the male pronucleus of fertilized eggs from C57BL/6J×SJL F2. Injected zygotes were implanted into pseudopregnant recipients, and a total of 49 potential FO founders were produced. A total of 8 positive founders were identified using tail DNA by PCR and Southern blot methods. These positive founders were backcrossed with wild type C57BL/6J mice. In order to obtain the transgenic line with high expression of hIL-1β in stomach tissues, the F1 pups from 5 positive founders were further screened by determining the concentration of hIL-1β in stomach tissues using the ELISA method. One transgenic line was obtained that highly expressed hIL-1β (line 19) and two lines were obtained that that weakly expressed hIL-1β in the stomach (line 24 and line 42) (FIG. 1B). Line 19 and line 42 were chosen to mate for further analysis.

This Example further shows that hIL-1β was specifically overexpressed in the stomachs of IL-1β transgenic mice. The concentration of hIL-1β in the stomach and serum of F5 pups using the ELISA method. The results showed the concentration of hIL-1β in the stomach tissues was significantly higher in the IL-1β transgenic mice than in the control mice. The concentration of human IL-1β was significantly higher in the stomach tissues than that in the serum of transgenic mice (FIG. 1C). Furthermore, the level of IL-1β in the stomach was higher in 4-month young mice than in the 12-month older mice (FIG. 1C). The concentration of IL-1β in the stomach tissues of line 19 mice was significantly higher in line 42 (FIG. 1C). The results also showed that the concentration of hIL-1β in the kidney tissues was not significantly different between transgenic mice and control mice.

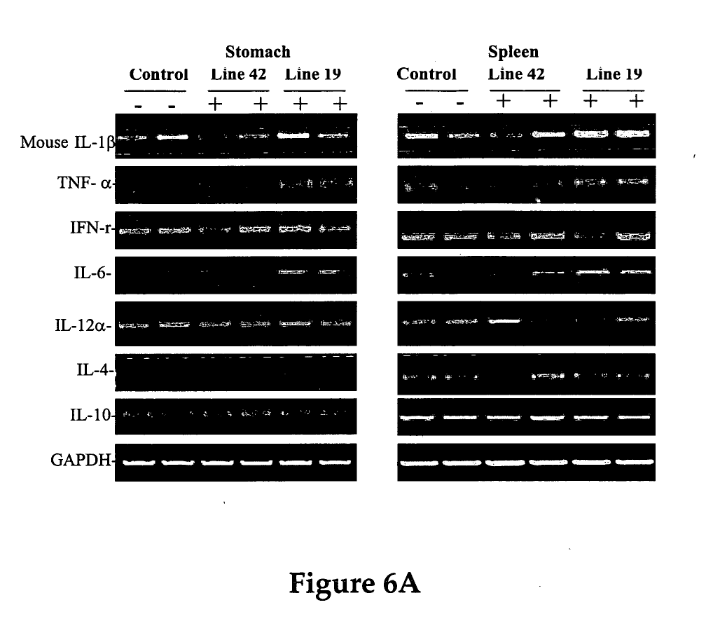

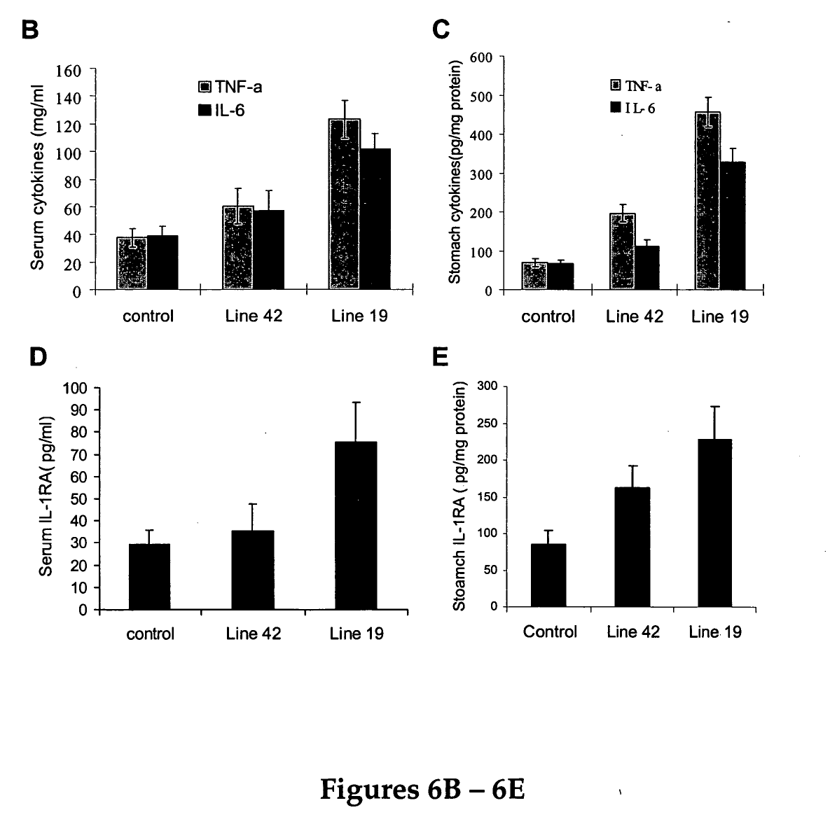

Moreover, mRNA expression of hIL-1β in the stomach tissue was significantly higher in line 19 mice than in line 42 mice (FIG. 1D). The expression of mouse IL-1β was not significantly changed between line 19 mice and control mice. The mRNA expression of hIL-1β was significantly higher in the stomach tissues of line 19 transgenic mice than in the spleen tissues. There was not a significant difference in the mRNA expression of mouse IL-1β between IL-1β transgenic mice and control mice (FIG. 1D). No mRNA expression of hlL-1β was detected in the stomach tissues of control mice and spleen tissues in both transgenic and control mice. The expression of mouse IL-1β was slightly elevated in the stomach and spleen in line 19 mice than in control mice (FIGS. 1D and 6A). These results show that hIL-1β was targeted to and expressed in stomach tissue by an H/K-ATPase promoter.

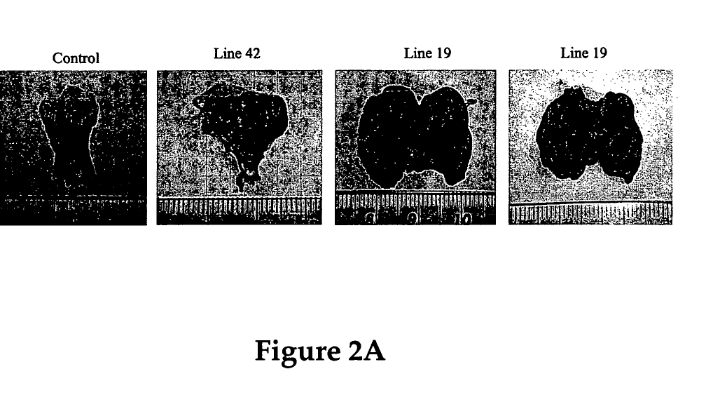

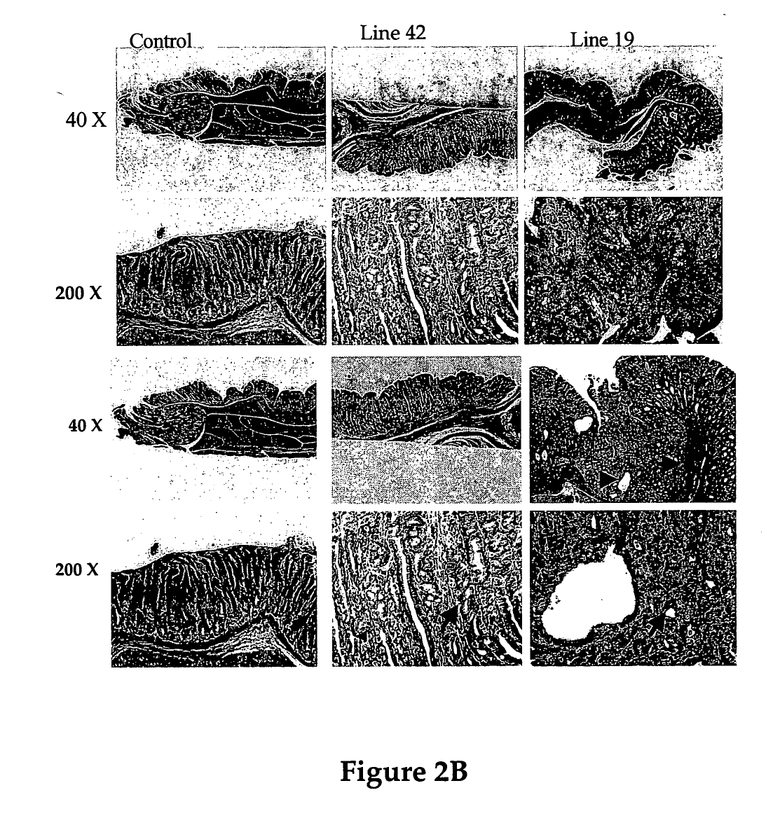

IL-1β transgenic mice spontaneously develop gastric inflammation and dysplasia. The transgenic and control mice were sacrificed at different ages between 2 and 20 months. FIG. 2A shows that IL-1β transgenic mice more than 1 year old exhibited marked larger size of stomach and thicker gastric folds than control mice. Grossly, the hypertrophy involved only the fundus of the stomach and resulted in thickened gastric folds, whereas the gastric antrum appeared uninvolved (FIG. 2B). The ratio of stomach weight/body weight was significantly higher in line 19 (2.13%±0.09%) than in control mice (0.79%±0.07%) (p<0.05) (Table 2) and was also slightly higher in line 42 (1.17%±0.34%) than in control mice (0.79%±0.07%), but the difference was not statistically significant. The results showed high expression of IL-1β in transgenic mice caused the significant increase in size of stomach.

TABLE 2
Overexpression of IL-1 β increased the ratio
of stomach weight/body weight

| | Num- | | | Stomach/Mice | |
|---|---|---|---|---|---|
| Line | ber | Mice (g) | Stomach (g) | body | P |
| Control | 8 | 31.45 ± 1.35 | 0.247 g ± 0.04 | 0.79% ± 0.07% | |
| Line 42 | 12 | 33.1 ± 3.1 | 0.359 ± 0.15 | 1.17% ± 0.34% | >0.05 |
| Line 19 | 15 | 38.2 ± 5.2 | 0.820 ± 0.14 | 2.13% ± 0.09% | <0.05 |

The transgenic mice and control mice at the age of 2-20 months were sacrificed. The mice and stomach were weighed. The results showed the average weight of mice body and stomach in each line.
P > 0.05, compared with the ratio of stomach weight/body weight of control mice;
P < 0.05, compared with the ratio of stomach weight/body weight of control mice.

The size of stomach was analyzed to determine the proliferation of stomach mucosa in IL-1β transgenic mice. The results showed that IL-1β transgenic mice more than 1 year old exhibited marked gastric hypertrophy. Grossly, the hypertrophy involved only the fundus of the stomach and resulted in thickened gastric folds, whereas the gastric antrum appeared uninvolved (FIGS. 2A and 2B). The hypertrophy of the oxyntic glands was caused primarily by marked foveolar hyperplasia (FIGS. 2A and 2B).

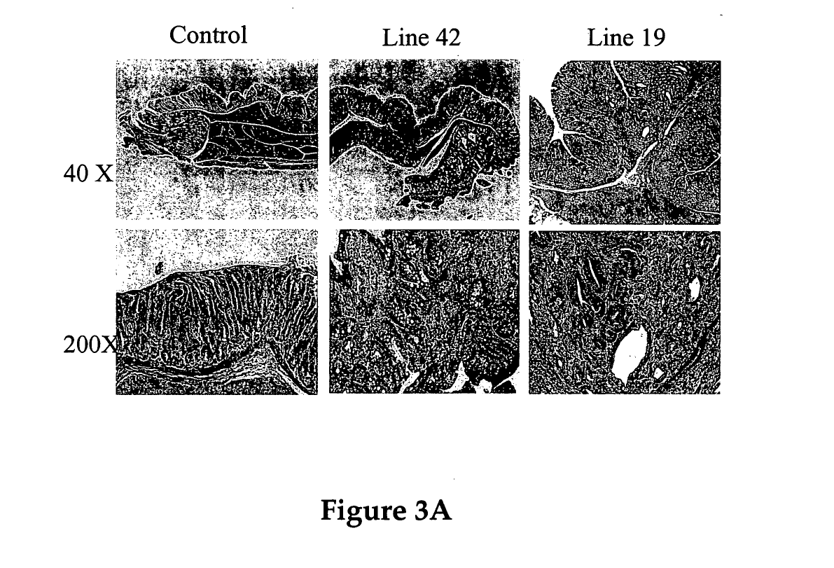

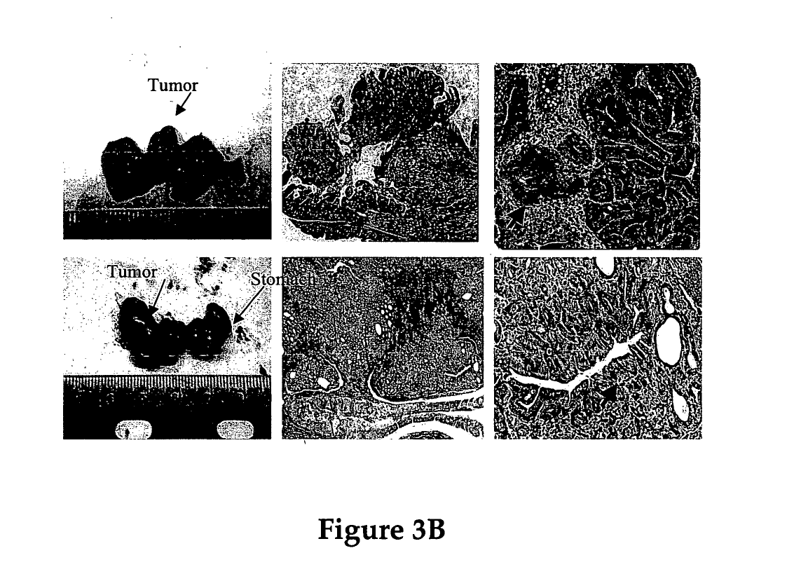

Pathological changes were further analyzed by H&E staining. All line 42 transgenic mice more than 1 year old developed hyperplasia and inflammation, 20% female and 37% male mice developed atrophy and metaplasia (FIGS. 2B and 3A). 25% of male mice developed dysplasia, but no female mice developed dysplasia. In addition, some line 42 mice exhibited the morphologic changes of hyalinosis and epithelial defects (Table 3).

TABLE 3
The changes of inflammation and pathology in the body of stomach of IL-beta mice

| | | | | Epithelial | Glandular | Acute | Chronic | Oxnt | Mucous | | | Lymphoid |
|---|---|---|---|---|---|---|---|---|---|---|---|---|
| | Sex | No. | Hyalinosis | defects | Hyperplasia | Inflam. | Inflam. | Atrophy | Metaplasia | Dysplasia | Cancer | follicles |
| Control | F | 4 | 0 | 0 | 0 | 0 | 0 | 0 | 0 | 0 | 0 | 0 |
| | M | 4 | 0 | 0 | 0 | 0 | 0 | 0 | 0 | 0 | 0 | 0 |
| Line 42 | F | 8 | 0 | 40% | 100% | 100% | 100% | 20% | 20% | 0 | 0 | 0 |
| | M | 10 | 20% | 42% | 100% | 100% | 100% | 37% | 37% | 25% | 0 | 30% |
| Line 19 | F | 13 | 33% | 77% | 100% | 100% | 100% | 77% | 77% | 67% | 0 | 77% |
| | M | 15 | 50% | 90% | 100% | 100% | 100% | 100% | 100% | 100% | 20% | 90% |

Note:
The section of stomach were stained with H&E. The pathological changes in the gastric corpus were analyzed according to the diagnosis criterion described in Material and Methods.

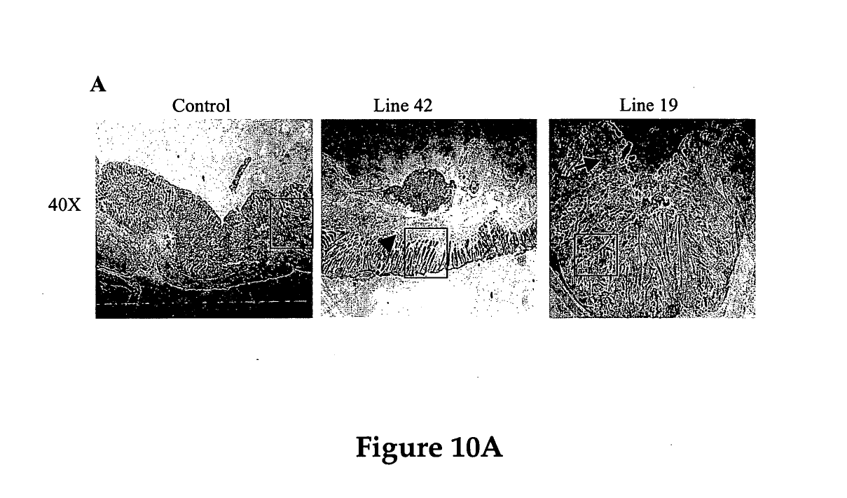

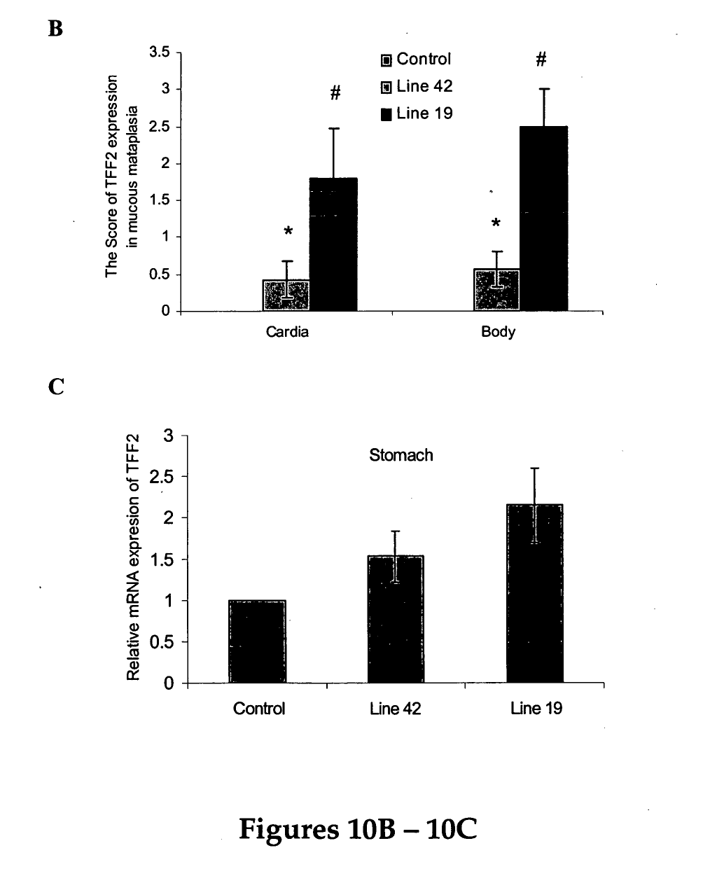

Microscopically, the gastric mucosa were thicker in IL-1β transgenic mice than in control mice. Gastritis was obviously observed in all transgenic mice and inflammatory infiltrate consisted of a mixed population of mononuclear and polymorphonuclear leukocytes, partly with lymphoid follicles (FIGS. 2B and 3A). Hyperplastic branching tortuous glands and glandular enlargement were observed in the stomachs of IL-1β transgenic mice. In line 19 mice more than 1 year old, all mice developed more severe hyperplasia, inflammation, and metaplasia, and both male and female mice developed dysplasia. More line 19 mice had the morphologic changes of hyalinosis and epithelial defects as well as lymphoid follicles infiltration (Table 3). Moreover, the inflammation and pathological scores were significantly higher in IL-1β transgenic mice than in control mice, and were significantly higher in line 19 mice than in line 42 mice (Table 4). Furthermore, the aberrant expression of TFF2/SP-positive cells, a characteristic of premalignant conditions, were found in mucus metaplasia area of the stomach in IL-1β mice, while TFF2 was only expressed in neck cells of the stomach in control mice (FIGS. 10A-10C). The scores of TFF2 expression in mucous metaplasia were significantly higher in the Line 19 IL-1β mice than in the Line 42 mice (Table 4), and the level of TFF2 mRNA was significantly higher in IL-1β mice than in control mice (FIGS. 1B and 1C).

TABLE 4
The inflammation score of body of stomach in IL-1beta mice

| | Glandular | Acute | Chronic | Oxyntic | Mucous | |
|---|---|---|---|---|---|---|
| | hyperplasia | inflammation | inflammation | Atrophy | Metaplasia | Dysplasia |
| | X ± SD | X ± SD | X ± SD | X ± SD | X ± SD | X ± SD |
| Control | 0 | 0 | 0 | 0 | 0 | 0 |
| Line 42 | 0.61 ± 0.70* | 0.61 ± 0.92* | 0.17 ± 0.38* | 0 | 0.33 ± 0.59* | 0.17 ± 0.38* |
| Line 19 | 2.0 ± 0.89#* | 1.47 ± 0.54#* | 2.0 ± 0.71* | 1.07 ± 0.7#* | 1.60 ± 0.84#* | 1.40 ± 0.83* |

Note:
Sections of stomach from transgenic and control mice were stained with H & E. The scores of inflammation and pathology were graded according the diagnosis criterion described in Material and Method. The results was presented the average of two times independent analysis.
Vs Control, *p < 0.01;
Vs Line 42 #p < 0.01

More importantly, 20% (3/15) line 19 male mice developed gastric carcinoma (FIGS. 3B and Table 3). These results showed that male mice were more susceptible to develop dysplasia and carcinoma. These results clearly show that overexpression of IL-1β directly caused gastritis and dysplasia (carcinoma).

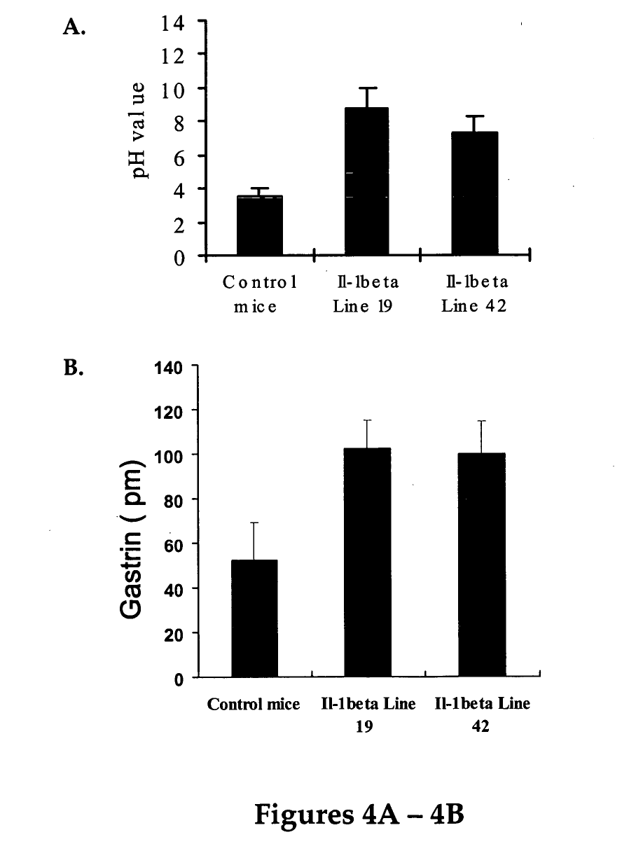

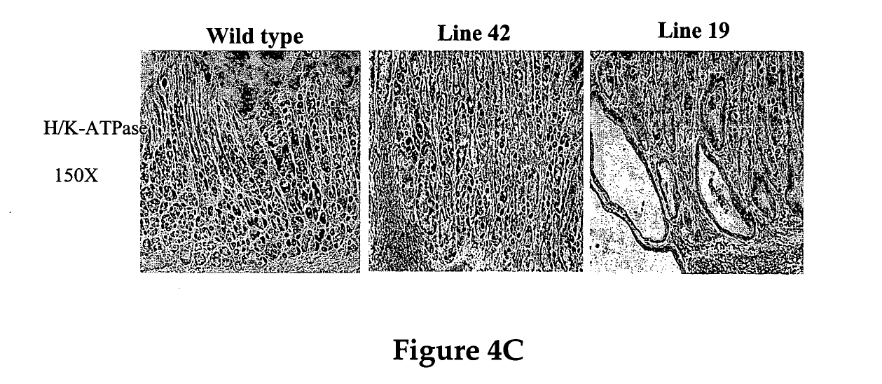

Overexpression of IL-1β reduces gastric acid secretion and loss of parietal cells. The mechanism in which IL-1β induced inflammation and carcinogenesis was investigated in transgenic mice. Since IL-1β is a powerful inhibitor of gastric acid secretion. Lower gastric acid may play a role in stomach carcinogenesis. It was determined whether endogenous overexpression of IL-1β inhibits the secretion of acid. The pH value of gastric juice was measured in transgenic mice. Results showed that the pH value of gastric juice was significantly higher in the IL-1β mice than in the control mice (p<0.05) (FIG. 4A), while the level of serum gastrin was higher in the IL-1β mice than in the control mice (FIG. 4B). It was further investigated whether the reduction of the acid secretion was due to the change of parietal cells in the stomach tissues. The results from the immunohistochemical staining using H/K-ATPase antibody showed that the number of parietal cells in the stomach tissues of the IL-1β mice significantly decreased, compared with that in the control mice. The decrease in the number of parietal cells was more significant in the areas of atrophy, dysplasia and carcinoma in the stomach (FIG. 4C). The results showed that the development of gastric carcinoma accompanied the loss of parietal cells in the IL-1β mice.

Since IL-1β is also a potent inhibitor of gastric acid secretion, the effect of endogenous overexpression IL-1β in stomach on gastric acid secretion was evaluated. The results showed that the pH value was significantly higher in the IL-1β mice than control mice (FIG. 4A), indicating that endogenous overexpression of IL-1β inhibited the gastric acid secretion in the transgenic mice. The effect of endogenous overexpression IL-1β on gastrin secretion was also analyzed. The results showed that endogenous overexpression of IL-1β significantly increased the level of gastrin in the IL-1β transgenic mice than in the control mice. No significant difference was detected in the level of gastrin between the line 19 mice and the line 42 mice (FIG. 4B).

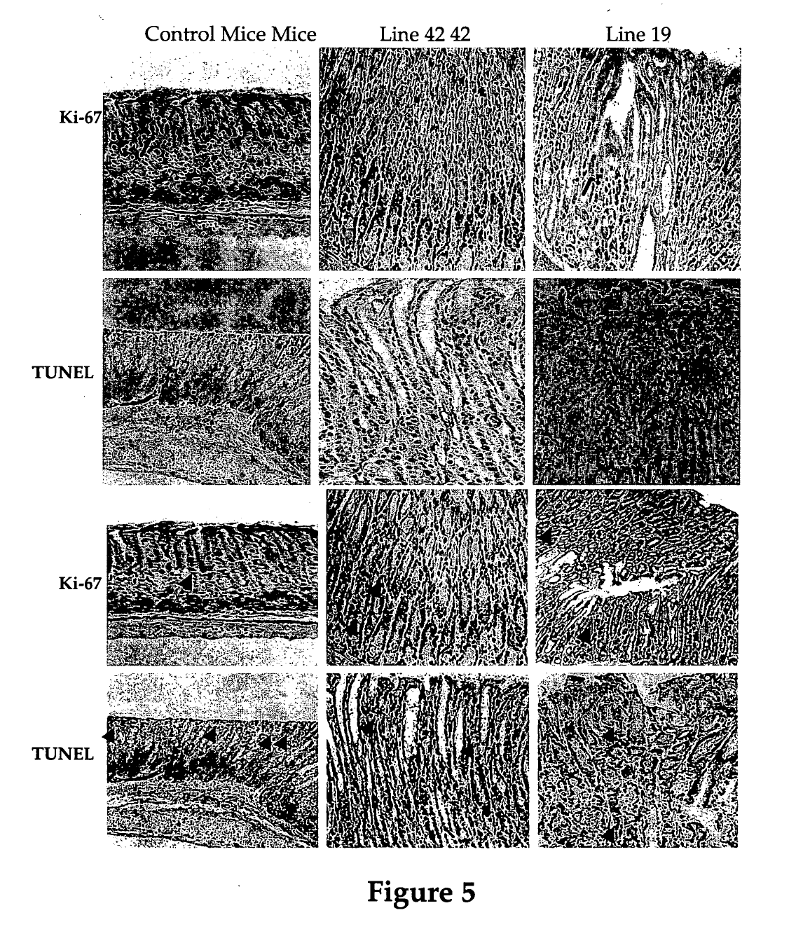

Overexpression of IL-1β causes the imbalance of cell proliferation and apoptosis in the stomach. The balance of cell proliferation and cell apoptosis plays a role in carcinogenesis. Thus, changes of cell proliferation and apoptosis in the stomach tissues of transgenic mice were determined. The number of ki-67 positive cells was higher in both lines of transgenic mice compared with the age-matched control mice. The ki-67 positive cells were located in the neck cell region of the gastric glands (FIG. 5). The apoptotic cells were located in the surface epithelial area in the control mice. The number of apoptotic cells in the surface epithelial area was significantly decreased in the transgenic mice compared to control mice. However, more apoptotic cells were observed in the gland cells of IL-1β mice (FIG. 5). It was possible that part of the parietal cells developed apoptosis. The results suggest that overexpression of IL-1β causes an imbalance between cell proliferation and apoptosis in the stomach.

Overexpression of IL-1β increases the production of proinflammatory factors. Since IL-1β is a proinflammatory factor, it was determined whether endogenous overexpression of IL-1β increased the product of proinflammatory factors such as cytokines. Measurements were made of the mRNA expression of Th1 cytokines IL-1β, TNF-α, IFN-γ, IL-6 and Th2 cytokines IL-4 and IL-10 in the stomach tissues and spleens. The representative results obtained using RT-PCR are presented in FIG. 6A. The results showed that the mRNA expressions of TNF-α and IL-6 were significantly increased in the stomach tissues of line 19 mice, compared with that in the control mice. Slight increases in mRNA expression of TNF-α and IL-6 were found in the stomach of line 42 mice compared with that in control mice. Also, the mRNA expression of TNF-α and IL-6 increased in spleen of IL-1β mice compared with control mice. Real time PCR confirmed that the mRNA expressions of TNF-α and IL-6 in the stomach and spleen were higher in the transgenic mice than in the control mice. However, the expression of IL-4 and IL-10 in the stomach and spleen was not significantly changed between transgenic mice and control mice. These results indicate that overexpression of IL-1β promoted inflammation and activated the Th1 cytokines immune response.

To investigate the role of TNF-α and IL-6 in IL-1β-induced inflammation and dysplasia, the concentration of IL-6 and TNF-α in the stomach and serum was determined. The results showed that the stomach and serum level of TNF-α in IL-1β mice was significantly increased compared with control mice (p<0.005) (FIGS. 4B and 4C). Although the concentration of TNF-α in line 19 IL-1β mice was slightly higher than that in line 42 mice, but no statistical difference (p>0.05). Also, the serum level of IL-6 in IL-i mice significantly increased compared with control mice (p<0.005). Although the concentration of IL-6 in Line 19 IL-1β mice was slightly higher than that in line 42 mice, but no statistical t difference (p>0.05). The level of IL1-RA in stomach and serum was significantly higher in IL-1β mice than in control mice (FIGS. 4D and 4E). The high level of IL-1RA may be a feedback result from high levels of IL-1β. These results suggest that TNF-α and IL-6 are the downstream targets of IL-1β, and may play an important role in IL-1β-induced inflammation and carcinogenesis.

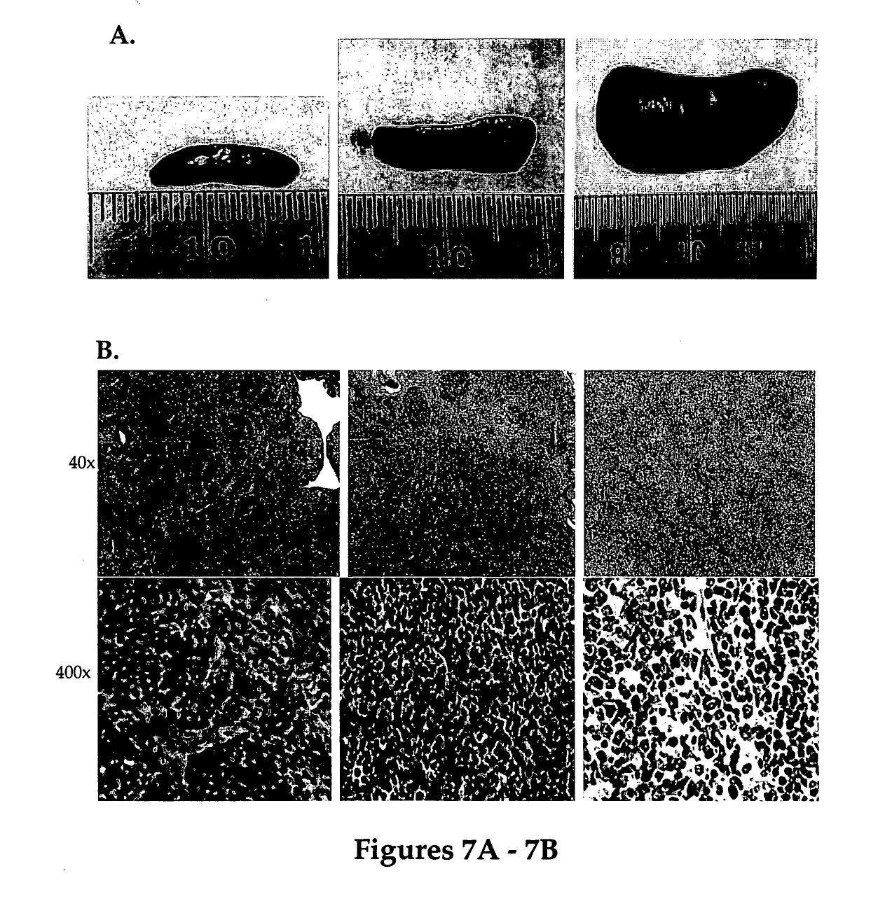

Overexpression of IL-1β causes inflammatory response and immune imbalance. In a preliminary experiment, continuous infusion (13 days) of IL-1β into C57BL/6J mice caused gastric atrophy, but not in Balb/c mice, indicating that a Th1 immune response may play a role in gastric atrophy induced by IL-1β. The results showed that the spleens were larger in IL-1β transgenic mice than in control mice (FIG. 7A and Table 5).

TABLE 5
Overexpression of IL-1 β increased the ratio of
spleen weight/body weight

| | | | |
|---|---|---|---|
| Line | Number | Spleen/body weight | P |

| | | | | |
|---|---|---|---|---|
| Control | 8 | 0.25% | 0.08% | |
| Line 42 | 12 | 0.43% | 0.03% | >0.05 |
| Line 19 | 15 | 0.88% | 0.04% | <0.05 |

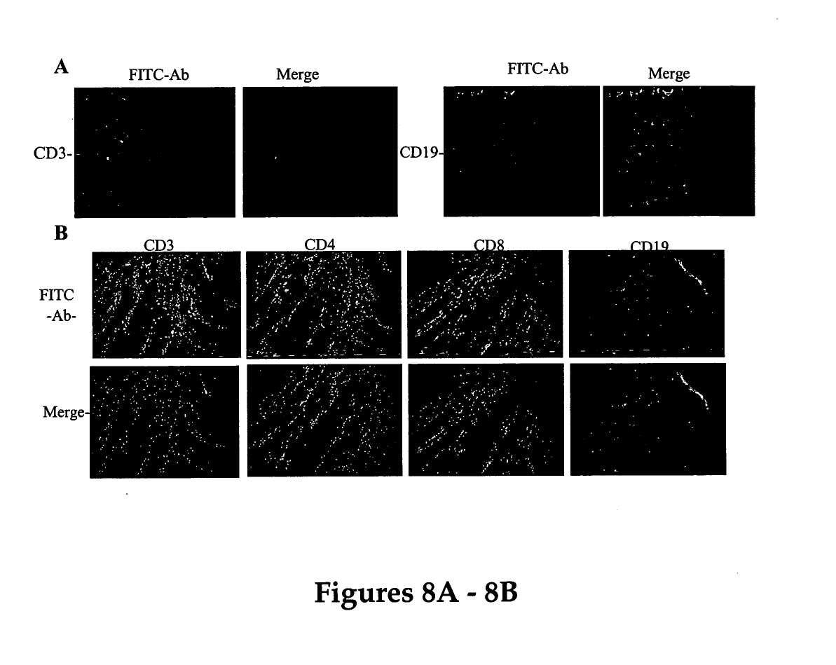

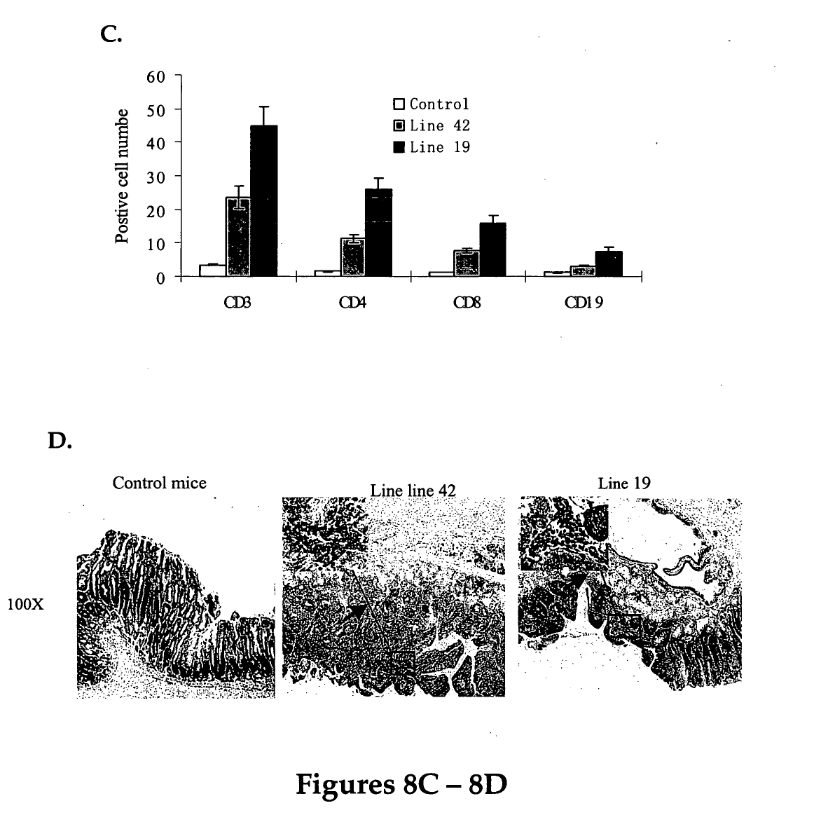

The expression of T and B lymphocytes was directly detected in the stomach tissues of transgenic mice. Representative sections stained with hemotoxylin and eosin confirmed hypertrophy and presence of mononuclear infiltrates within the gastric mucosa of IL-1β mice compared with control mice. Sections were stained to various cell surface markers specific for CD3⁺ T cells CD4⁺ T cells, CD8⁺ T cells, CD19⁺B cells and visualize DAPI staining. There was a significant increase in the number of CD3⁺, CD4⁺ and CD8⁺ T cells in the mucosa of stomach in IL-1β mice, very few B cells were found in the mucosa of stomach in IL-1β mice (FIGS. 8A and 8B). The quantity assay showed that the number of CD4⁺ T cells was higher than that of CD8⁺ cells (FIG. 8C). However, a few B cells were observed in follicle-like aggregates (FIG. 8B). Very few positive staining was observed for CD3⁺ T cells and CD 19⁺ B cells in the stomach of control mice (FIG. 8C). The data indicated that the proliferation and activation of T cells may play an important role in gastric inflammation and dysplasia. Furthermore, NF-κB RelA (p65) nuclear staining was also higher in the inflammatory cells of the stomach in IL-1β transgenic mice, no p65 staining was observed in the stomach of control mice, suggesting that NF-κb activation plays an important role in inflammation and carcinogenesis in IL-1β mice (FIG. 8D).

Moreover, the IL-1β transgenic mice have developed the splenomegaly (FIG. 7A). The ratio of spleen weight/body weight was significantly higher in line 19 mice (0.88%±0.04%) and in line 42 (0.43%±0.03%) than in control mice (0.250%±0.08%). However, pathological changes were not observed in other tissues including liver, colon, small intestine, kidney and lung. The results showed high expression of IL-1β induced-inflammatory and immune response resulted in splenomegaly in transgenic mice. Pathological analysis showed that the spleen tissues of the transgenic mice exhibited the marked hyperplasia in IL-1β transgenic mice, and that a large number of inflammatory infiltrations with leukocytes, neutrophils and macrophages were observed in the spleen tissues of transgenic tissues (FIG. 7B and Table 4). The results indicated that IL-1β transgenic mice developed severe inflammatory and immunity response. Changes of T and B lymphocyte subpopulations were further determined in the spleen from transgenic mice by FACS. The results showed that CD3, CD3/CD8, CD3/CD44 and CD11b were significantly higher in line 19 mice than in control mice, and slightly higher in line 42 mice than in control mice, respectively, while the percentage of CD19 was significantly lower in line 19 IL-1β, mice than in control mice, and slightly lower in line 42 IL-1β mice than in control mice (Table 6). Thus, The data strongly suggest that overexpression of IL-1β-induced immunity imbalance may play a pivotal role in the development of inflammation and dysplasia in IL-1β transgenic mice.

TABLE 6
Changes of splenic lymphocytes in IL-1 β mice

| | Wt | Line 19 | Line 42 |
|---|---|---|---|
| CD3 | 25.7 ± 2.1 | 35.8 ± 6.8* | 29.70 ± 7.3 |
| CD3/CD4 | 13.4 ± 0.5 | 16.3 ± 6.7 | 18.4 ± 6.7 |
| CD3/CD8 | 6.2 ± 0.1 | 16.4 ± 5.4* | 8.3 ± 5.5 |
| CD19 | 56.3 ± 1.2 | 29.4 ± 2.4* | 37.0 ± 9.8 |
| CD116 | 7.2 ± 1.8 | 17.9 ± 7.0* | 8.3 ± 5.7 |
| CD11C | 4.7 ± 0.5 | 7.4 ± 2.6 | 7.0 ± 2.4 |
| CD3/CD25 | 1.8 ± 0.1 | 2.1 ± 0.7 | 1.8 ± 0.6 |
| CD3/CD44 | 15.5 ± 1.5 | 39.1 ± 12.1# | 26.3 ± 12.9 |

Note:
spleen cells were isolated and subjected stained with specific antibodies as indicated. The cells was analyzed by FACS. The results are the average from 4 mice.

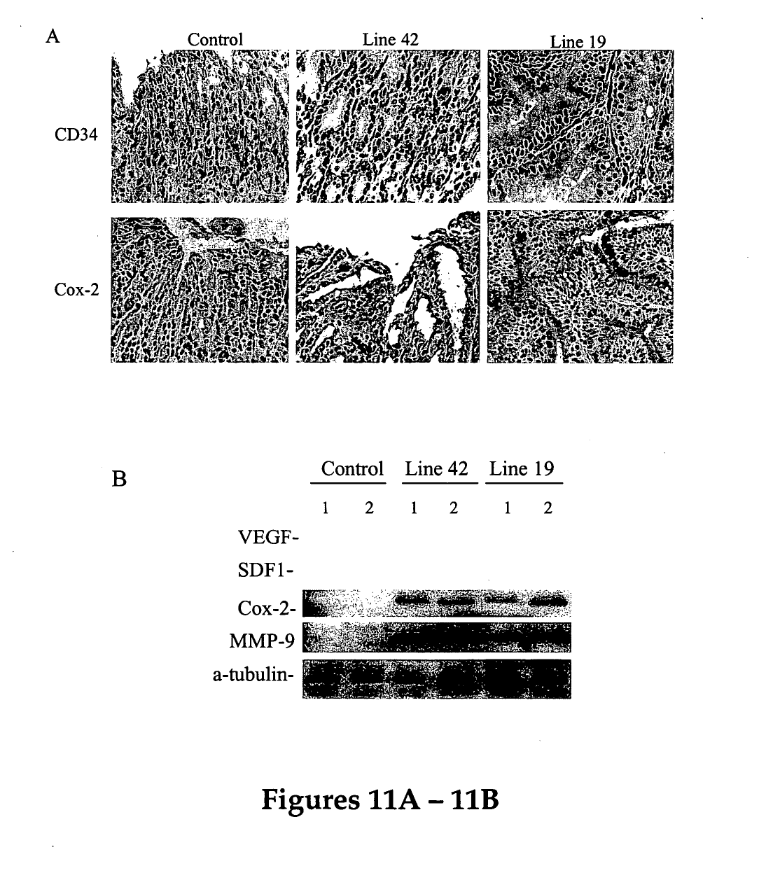

Overexpression of IL-1β increases angiogenesis in the stomach tissue. Inflammatory angiogenesis is a critical process in tumor progression. IL-1β modulates angiogenesis by directly interacting with vascular endothelial cells or enhancing the production of proangiogenic factors via paracrine. Angiogenesis was investigated in the stomach tissues of IL-1β mice. The IL-1β-induced angiogenesis in vivo was determined by staining CD34 antibody. The IL-1β-induced angiogenesis in the stomach was significantly increased in the IL-1β mice compared with control mice (FIG. 11A). Some reports showed that IL-1β induced angiogenesis in vitro and in vivo through the COX-2-prostanoid pathway, also involved in the upregulation of VEGF and MMPs. Expression of COX-2, VEGF and MMPs was determined in the stomach tissues. Immunohistochemical staining showed that Cox-2 was intensively expressed in the dysplasia areas of stomach tissues in IL-1β, mice, whereas rare Cox-2 positive staining was observed in the stomach of control mice (FIG. 11A). The results were confirmed by Western blot (FIG. 11B). The expressions of VEGF were significantly increased in the stomach tissues in IL-1β mice than in control mice (FIGS. 11A and 11B). The expressions of MMP-9 protein were also significantly increased in the stomach tissues in IL-1β mice than in control mice (FIGS. 11A and 11B). These results show that transgenic expression of IL-1β increases angiogenesis in the stomach tissue partly by upregulation of Cox-2, VEGF and MMP-9 expression.

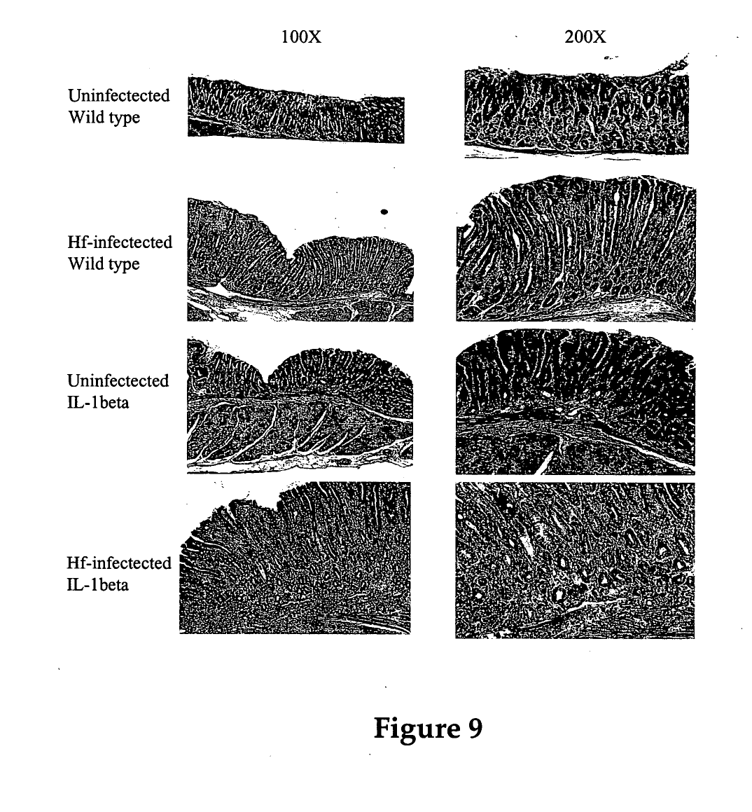

Helicobacter felis infection accelerated the development of gastritis and carcinoma in IL-1β mice. Patients with IL-1 genetic polymorphisms and H. pylon infection have higher risk in the development of gastric cancer. Thus, it was further studied whether Helicobacter felis infection accelerated the development of gastritis and carcinoma in IL-1β mice. The effect of a short-term infection of H. felis on the development of gastritis in IL-1β mice was analyzed. The results showed that the IL-1β mice infected with H. felis developed more severe gastric inflammation compared with infected wild type mice and uninfected IL-1β mice (FIG. 9).

Helicobacter felis infection increases the mobilization of hematopoietic stem cells into the circulation and stomach tissues. Previous results showed that gastric cancer originates from bone marrow-stem cells, therefore it was investigated whether overexpression of IL-1β induced the mobilization hematopoietic stem cell (HSCs), and whether HSC recruitment contributes to the development of dysplasia. GFP+ bone morrow was transplanted from GFP transgenic mice into IL-1β and C57B/6J mice and the number of hematopoietic stem cells in blood, spleen and stomach was determined. It was further found that IL-1RA treatment reduces the development of gastritis and the mobilization of hematopoietic stem cells circulation and stomach tissues.

Discussion

Gastric carcinogenesis is a multistep and multifactorial pathological process. The environmental factors, host factor and bacterial virulence are likely to be involved in pathogenesis. H. pylori infects has been confirmed in the pathogenesis of gastric cancer. Approximately 20% of Helicoobacter pylori-infected individuals develop clinically significant diseases such as peptic ulcer, gastric adenocarcinoma, or gastric mucosa-associated lymphoid tissue (MALT) lymphoma (13,27). However, It is unknown which bacterial, host, and environmental factors are the critical determinants that predispose to these clinical manifestations of H. pylori infection. Recently, a host genetic factor, IL-1β gene has been proposed as a key factor in determining the pattern of gastritis and risk of malignant transformation (19). Host genetic factors have recently been focused on as the reasons for the divergent clinical outcomes by H. pylori infection. IL-1β is one of the representative proinflammatory cytokines induced by H. pylori infection. IL-1β promotes the development of gastric cancer induced by MNNG in rodents (28). Therefore, IL-1β is now thought to have an important part in the development of H. pylori-related gastric cancer (19). The large numbers of clinical epidemiological studies have suggested that IL-1 genetic polymorphism were associated with increased risk of gastric cancer (20-21). The IL-1 gene cluster (IL-1B-511T/-31 C and IL-IRN*2/*2) influenced H. pylori-related gastric mucosal IL-1β levels and were related to hypochlorhydria, gastric inflammation, atrophy and carcinogenesis (20-22). However, some data are conflicting regarding the effects of these polymorphisms on IL-1β production (29,30), indicating that gastric cancer risk is also associated with many genetic factors (including IL-1β, IL-6, IL-10, TNF-□, GST, NAT, Cytochrome P450 isoenzymes, MUCI) as well as a variety of environmental factors (e.g., salt intake and smoking) (30,31). Thus some key questions about the effect of IL-1β in gastric cancer remain to be elucidated.

This Example shows for the first time that human IL-1β can be specifically expressed in mouse stomach tissue through the mouse H/K-ATPase promoter. Overexpression of a constitutively secreted form of IL-1β leads to gastric inflammation, resulting in gastritis and hypochlorhydria, followed by progression to gastric atrophy, metaplasia, dysplasia and cancer. There is a dose-response relationship with a more pronounced phenotype in the high-expressing line (line 19) compared to the low expressing line (line 42). Despite the targeted expression of IL-1β to only the stomach, the transgenic mice showed evidence of systemic immune activation with splenic hyperplasia and increased numbers of macrophages, NK cells, and activated T cells. Overexpression of IL-1β in the stomach led to increased circulating levels of IL-6 and TNF-α. Infection of IL-1β transgenic mice with H. felis led to accelerated progression to atrophy and cancer. This Example demonstrates that dysregulated expression of a single cytokine may be sufficient for induction of cancer.

This Example further demonstrates that endogenous overexpression of human IL-1β induced spontaneous gastritis and carcinoma even absent the Helicobacter infection using a IL-1β transgenic mouse model. IL-1β transgenic mice more than one year old developed gastric hypertrophy with larger size and thicken folds. Pathological analysis showed that the transgenic mice spontaneously developed the hyperplasia, acute and chronic inflammation and dysplasia. The hyperplasia of stomach was due to the higher rate of cell proliferation and lower rate of apoptosis in the epithelial cells and gland cells of stomach tissues in the transgenic mice. Interestingly, these pathological changes mainly occurred in the corpus of stomach but not in the antrum. This was consistent with the expression pattern of human IL-1β in the parietal cells of stomach body, indicating that endogenous overexpression of IL-1β directly induces gastritis and dysplasia in the corpus of stomach. Furthermore, the line 19 transgenic mice with higher level of hIL-1β developed more severe acute and chronic gastritis, metaplasia, dysplasia than line 42 mice with a lower level of hEL-1β. More importantly, male mice of line 19 developed spontaneous stomach cancer. The results clearly showed that the degree of inflammation and dysplasia were directly related with the level of IL-1β in the stomach. Male mice were susceptive to develop dysplasia and carcinoma. These results were consistent with the clinical data that IL-1β polymorphisms with high production of IL-1β have a higher risk of gastric cancer in men than in women (22).

High gastric expression levels of IL-1β were achieved using the hIL-1β cDNA fused to the signal peptide for IL-1RA. Previous studies had demonstrated that this strategy resulted in high levels of expression when targeted to the spleen, leading to altered lymphocyte differentiation (24). Other studies have shown that overexpression of a non-mutated form of the hIL-1β cDNA could achieve lower levels of hIL-1β. However, in the case of IL-1β, there appears to be a very narrow window between physiologic and pathophysiologic levels of expression, and IL-1β is thus a tightly regulated cytokine, with multiple mechanisms to limit expression of this toxic cytokine. Although elevated circulating levels of IL-1β were not observed, increased circulating levels of downstream cytokines (TNF-α and IL-6) were observed, and previous studies have suggested that IL-6 levels often are a surrogate for elevated IL-1β activity. In addition, elevated levels of IL-1RA, the natural circulating antagonist, were observed, but despite the increase in the circulating antagonist, elevated levels of IL-1β still led to a marked phenotype.

One of critical factors in gastric carcinogenesis is in gastric acid secretion. Gastritis that is confined to the antral region is associated with excessive acid production and a high risk of duodenal ulcer disease (32). In contrast, gastritis involving the acid-secreting corpus region leads to hypochlorhydria, progressive gastric atrophy and an increased risk of gastric cancer (33). Host factors that influence acid secretion potentially influence the outcome of an H. pylon infection. IL-1β is a powerful inhibitor of gastric acid secretion (17). IL-1β produced locally in the gastric mucosa is one of the mediators of the inhibitory effects of H. pylori-induced inflammation on gastric acid secretion (18). Decreased acid secretion was accompanied by an elevation of IL-1β messenger RNA levels in the H. pylori-infected gastric mucosa, and the effect was reversed after injection of an IL-1β endogenous receptor antagonist (18). Furthermore, IL-1β is upregulated in the presence of H. pylori and is important in initiating and amplifying the inflammatory response to this infection (16). IL-1β polymorphisms increase both the likelihood of a chronic hypochlorhydric response to H. pylori infection and the risk of gastric cancer, presumably by altering IL-1β levels in the stomach (19). This functional inhibition of IL-1β on parietal cells is initially reversible but the progressive destruction of parietal cells eventually leads to irreversible hypochlorhydria (32). The results presented here show that the gastric acid secretion was significantly inhibited and accompanied with feedback increase in the serum level of gastrin in the transgenic mice than in control mice. The inhibition of acid secretion was due to the loss of parietal cells caused by high level of IL-1β. Since hML-1β was expressed and secreted by parietal cells, thus local high level of IL-1β in turn caused inflamed destruction to parietal cells and leads to hypochlorhydria (32). A decreased flow of gastric acid secretion may therefore heighten mucosal damage by allowing the accumulation of bacterial toxins and by-products of inflammation. While hypochlorhydria permits superinfection by other bacteria that enhance the production of highly carcinogenic N-nitroso compounds. Hypochlorhydria also markedly reduces the levels of vitamin C in gastric juice, further facilitating the formation of N-nitroso compounds. The results show that pathological changes occurred in the corpus of stomach of transgenic mice supported the clinical data that genotypes with high IL-1β production may favor the initiation of a set of responses to H. pylori that result in hypochlorhydria, corpus atrophy and an increased risk of gastric cancer.

The current study provides support and validation for previous studies that suggested a strong link between SNPs in the IL-1β gene and the risk of gastric cancer in the setting of H. pylori infection. IL-1β is upregulated to varying degrees in the presence of H. pylori and is important in initiating and amplifying the inflammatory response to this infection¹³. Multiple studies have now confirmed that a high-expressing IL-1β genotype is the major factor leading to atrophic gastritis and gastric cancer in the setting of H. pylori infection in human patients (14, 22, 55, 56), and this increased expression is link to specific polymorphisms in the IL-1 gene cluster (IL-1B-511T/-31 C and IL-IRN*2/*2). These genotypes have been associated with an increased risk of many types of cancer. In the H. felis mouse model, it is directly demonstrated that IL-1β overexpression results in more rapid progression to gastric atrophy and cancer. The current study indicates that overexpression of IL-1β is not only important but can be sufficient for induction of gastric cancer even in the absence of infection. This is the first cytokine to provide such a direct link between inflammation and cancer.

While IL-1β is likely important in the development of many cancers, it may have a particularly important role in stomach cancer, in part because of the strong effects on gastric physiology. Gastritis involving the acid-secreting corpus region leads to hypochlorhydria, progressive gastric atrophy and an increased risk of gastric cancer (32,33). IL-1β has previously been suggested to play a direct role in the inhibition of gastric acid, contributing to the development of hypergastrinemia (19,57). The results presented here showed that the gastric acid secretion was significantly inhibited and accompanied with an increased serum level of gastrin in the transgenic mice. However, the actions of IL-1β in the stomach include both a reversible inhibition of parietal cell function, as well as an irreversible and progressive destruction of parietal cells leading to gastric atrophy (32,33). In the cellular analysis of the IL-1β mice, increased gastric apoptosis was observed at early time points followed later by decreased numbers of parietal cells.

Besides inhibition of gastric acid, other mechanisms were involved in the development of gastritis and dysplasia in the IL-1β transgenic mice. IL-1β is a proinflammatory cytokine that itself cause inflammation (1). But more importantly, IL-1β induces the expression of other proinflammatory cytokines. Of major important are Cox-2, TNF-α, IL-6, iNOS and other cytokines/chemokines (34-36). This accounts for the rapid activation of stromal and immune cells to generate amounts of IL-1β, TNF-α and other cytokines. IL-1β and TNF-α stimulate their own and each other production. This represents an important amplification loop of the inflammatory response. Also, IL-1β and TNF-α increase the expression of adhesion molecules, such as intercellular adhesion molecule-1 (ICAM-1) which promotes leukocyte infiltration from the blood into tissues.

A number of studies have shown that the development of atrophy is primarily related to the nature of the host response to infection. With respect to gastric cancer, a strong Th1 response has been linked to the development of atrophy and cancer, while a Th2 immune response has been associated with protection from gastric cancer development, and shifting the immune response towards stronger adaptive Th2-polarized immune responses to H. pylori infection associated with enteric helminthes inhibits the development of cancer (25). In this study, the expression of Th1 cytokines were elevated in the IL-1β transgenic mice, whereas the expression of Th2 cytokines IL-4 and IL-10 in the stomach tissues and spleens did not changes, supporting the idea that chronic overexpression of IL-1β activated the Th1 cytokines immune response. It was previously shown that Helicobacter infection activates the innate immune system through a TLR2 pathway (58), leading to induction of NF-κB signaling and production by macrophages of cytokines (such as IL-1β, TNF-α and IL-6) encoded by target genes of the IKKβ-dependent NF-κB-activation pathway. In addition, macrophages have been strongly associated with the development and progression of cancer, with NF-κB providing the primary link. Nevertheless, the primary NF-κB-dependent genes in macrophages that can promote cancer have not been clarified. Studies suggest that IL-1β may be one of the critical macrophage-genes leading to cancer. IL-1 has the ability to autoregulate its own production, leading to its own upregulation as well as the release of numerous cytokines that can sustain NF-κB activation in macrophages. Increased upregulation of NF-κB expression was shown in the transgenic IL-1β mouse stomach, as well as upregulation of COX-2 and VEGF, other NF-κB target genes.

The loss of acid secreting cells and remodeling of the stomach with metaplastic cells is likely the result of immune activation. This includes macrophages as well as activated T cells, CD4+, CD44+ T cells. Previous studies have shown that IL-1β upregulation leads to a shift toward activated T cells in the spleen. The systemic immune activation and splenic hyperplasia (Table 7) was linked in some way to the development of cancer. In addition, mice showed elevated of circulating levels of EL-6 and TNF-α. IL-6 has often been used as a surrogate for IL-1β activity. IL-1β has been shown to promote the migration of activated macrophages and T cells.

TABLE 7
The pathological changes in the spleens of IL-1β mice

| | Sex | No. | Normal | Hyperplasia |
|---|---|---|---|---|
| Control | F | 4 | 100% (4/4) | 0 |
| | M | 4 | 100% (4/4) | 0 |
| Line 42 | F | 5 | 80% (4/5) | 20% (1/5) |
| | M | 7 | 71% (5/7) | 29% (2/7) |
| Line 19 | F | 7 | 43% (3/7) | 67% (4/7) |
| | M | 8 | 25% (2/8) | 75% (6/8) |

The IL-1β transgenic mice developed the severe inflammatory response with inflammatory infiltrate consisting of a mixed population of leukocytes, neutrophils and macrophages and partly with lymphoid follicles in the stomach and spleen tissue. These activated inflammatory cells increased the production of proinflammatory factors such as TNF-α and IL-6. High levels of TNF-α and IL-6 amplified the inflammatory response in the transgenic mice and accelerated gastric inflammation. Thus, TNF-α and IL-6 were the downstream targets of IL-1β, may play an important role in IL-1β-induced inflammation. Furthermore, only the expressions of Th1 cytokines were elevated in the IL-1β transgenic mice, but the expression of Th2 cytokines IL-4 and IL-10 in the stomach tissues and spleens did not change, indicating that overexpression of IL-1β promoted inflammation and activated the Th1 cytokine immune response.

Immune response undoubtedly has a significant impact on the potential for gastric carcinogenesis, and this is highlighted by the findings that severe combined immunodeficiency and T cell-deficient mice infected with Helicobacter do not development the same degree of tissues injury despite high levels of gastric bacterial colonization (37-38). The importance of T lymphocytes is also demonstrated by the experiments that showed that B cell-deficient Helicobacter-infected mice are not protected from severe atrophy and metaplasia (38). As a pleiotropic cytokine, IL-1β has diverse potentiating effects on the proliferation, differentiation, and function of diverse nonadaptive (NK cells, macrophages, granulocytes, etc.) as well as specific immunocompetent cells (T and B cells) (1,39). IL-1β also promotes hematopoiesis in the bone-marrow, mainly by its ability to increase the production of colony stimulating factors (CSFs) and stem cell factors (1). Of special relevance for tumor immunity are the effects of IL-1β on the activation of T cell-mediated immune responses. IL-1β is instrumental for the activation of both subsets of CD4⁺ T cells and CD8⁺ T cells. Consistent with the increase in Th1 cytokines, IL-1β transgenic mice exhibited significant increase in the total T cells, activated T cells, neutrophils and macrophages (CD11b⁺), decrease B cells in the spleen tissues. The CD11b is a marker of neutrophils and macrophages, and CD44 participates in the process of leukocyte recruitment to sites of inflammation and to their migration through lymphatic tissues and involved in T cells activation (40,41). Persistent inflammation, possibly intensified via the inflammatory cytokine cascade and the generation of specific T-cell immune responses, eventually leads to gastric atrophy, hypochlorhydria, and increased risk for developing gastric carcinoma. Thus, the results indicated that overexpression of IL-1β-induced the immunity imbalance may play a pivotal role in the development of inflammation and dysplasia in IL-1β transgenic mice.

The IL-1β transgenic mice showed increased levels of apoptosis which were followed later on by engraftment of BMDCs. These data suggest that IL-1β, overexpression leads to increased mobilization of progenitor cells from the bone marrow and spleen, followed by recruitment to the gastric mucosa. The recruitment of BMDCs was associated with upregulation of MMP-9 (using the MMP9-antibody treatment reduced the recruitment of BMDCs). Helicobater felis accelerated the development of gastric inflammation and increased mobilization of BMDCs. Whether overexpression of IL-1β-induced the recruitment of BMDCs are contributed to the development of dysplasia (carcinoma) and whether H. felis accelerated the process will be further investigated by long-term study.

A key factor in gastric carcinogenesis appears to be an early increase in apoptosis and proliferation leading to rapid tissue turnover that occur during the early phases of gastritis; in the severest cases, this results in the development of atrophy and metaplasia, and replacement of the stomach with cell lineages that are apoptosis resistant and thus premalignant.

Many studies have shown that IL-1β is involved in angiogenesis, tumorigenesis and invasion, markedly inducing angiogenesis in vitro and in vivo by upregulation of expression of various prostanoids (4,5). In microenvironmental stroma cells and in malignant cells, exogenous recombinant IL-1 induces secretion of growth and invasiveness-promoting factors, angiogenic factors (1). Endogenous and exogenous IL-1β markedly promoted tumor growth, increase tumor metastasis and induced angiogenesis in vitro and in vivo (6,8). The IL-1β induced angiogenesis in vitro and in vivo through the COX-2-prostanoid pathway. Accordingly, the results showed that transgenic overexpression of IL-1β increases angiogenesis and expression of Cox-2, VEGF and MMP-9. The inhibition of IL-1β by IL-1RA peptide or IL-1RA gene inhibited tumor growth and reduced angiogenesis and invasiveness (7,8). These data suggest that IL-1β plays an essential role in cancer development and may be a potential target of therapy using IL-1RA as a treatment.

These results provide the direct evidence that overexpression of IL-1β can induce spontaneous inflammation and dysplasia (carcinoma) even in the absence of Helicobacter pylori infection. IL-1β plays an important role in the development of gastric carcinogenesis, and represents a possible target for prevention and treatment of stomach cancer. The IL-1β transgenic mouse is a promising animal model to study the relationship among inflammation, cancer stem cell and gastric cancer.

### Example 2

### Generation of Transgenic Mouse Lines with Constitutive Expression of IL-1β in the Pancreas Tissue or Mammary Tissue

In order to study the role of the prototype cytokine, IL-1β, in cancer predisposition, the invention provides for two additional transgenic lines: one line expressing IL-1β in the pancreas acinar tissue, comprising a rat elastase-IL-1β transgene; and the other line carrying an MMTV-IL-1β transgene, designed to overexpress IL-1β in the mammary tissue. Both transgenes contain the identical recombinant human IL-1β cDNA with the IL-1RA signal peptide allowing the peptide to be constitutively secreted. Both transgenes (pancreas and mammary gland specific) were injected into the mouse germline and a minimum of 2-3 founder lines have been generated. Analysis of expression and phenotype will be achieved in future studies.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

### REFERENCES

1. Apte, R. N., et al. Interleukin-l a major pleiotropic cytokine in tumor-host interactions. Semin Cancer Biol 2002;12:277-290
2. Dinarello C. A. Biologic basis for interleukin-1 in disease. Blood 1996; 87: 2095-2147
3. P. E. Auron, The interleukin 1 receptor: ligand interactions and signal transduction. Cytokine Growth Factor Rev. 1998; 9: 221-237
4. Voronov E, Shouval D S, Krelin Y, Cagnano E, Benharroch D, Iwakura Y, Dinarello C A, Apte R N. IL-1 is required for tumor invasiveness and angiogenesis. Semin Cancer Biol 2002; 12:277-90.
5. Fan F, Stoeltzing O, Liu W, McCarty M F, Jung Y D, Reinmuth N, Ellis L M. Interleukin-1β Regulates Angiopoietin-1 Expression in Human Endothelial Cells. Cancer Res 2004;64:3186-90
6. Song X, Voronov E, Dvorkin T, Fima E, Cagnano E, Benharroch D, Shendler Y, Bjorkdahl O, Segal S, Dinarello C A, Apte R N. Differential effects of IL-1 alpha and IL-1 beta on tumorigenicity patterns and invasiveness. J Immunol 2003;171 :6448-56
7. McKenzie R C, Oran A, Dinarello C A, Sauder D N. Interleukin-1 receptor antagonist inhibits subcutaneous B16 melanoma growth in vivo. Anticancer Res 1996;16:437-44.
8. Vidal-Vanaclocha F, Amezaga C, Asumendi A, Kaplanski G, Dinarello C A. Interleukin-1 receptor blockade reduces the number and size of murine B16 melanoma hepatic metastases. Cancer Re 1994;54: 2667-2672
9. Voronov E, Shouval D S, Krelin Y, Cagnano E, Benharroch D, Iwakura Y, Dinarello C A, Apte R N. IL-1β is required for tumor invasiveness and angiogenesis. Proc Natl Acad Sci U S A. 2003;100:2645-50.
10. Vidal-Vanaclocha F, Fantuzzi G, Mendoza L, Fuentes A M, Anasagasti M J, Martin J, Carrascal T, Walsh P, Reznikov L L, Kim S H, Novick D, Rubinstein M, Dinarello C A. IL-18 regulates IL-1beta-dependent hepatic melanoma metastasis via vascular cell adhesion molecule-1. Proc Natl Acad Sci USA. 2000;97:734-9.
11. Carrascal M T, Mendoza L, Valcarcel M, Salado C, Egilegor E, Telleria N, Vidal-Vanaclocha F, Dinarello C A. Interleukin-18 binding protein reduces b16 melanoma hepatic metastasis by neutralizing adhesiveness and growth factors of sinusoidal endothelium. Cancer Res 2003; 63:491-7.
12. Weinreich D M, Elaraj D M, Puhlmann M, Hewitt S M, Carroll N M, Feldman E D, Turner E M, Spiess P J, Alexander H R. Effect of interleukin 1 receptor antagonist gene transduction on human melanoma xenografts in nude mice. Cancer Res 2003;63:5957-61.
13. Houghton J, Wang T C. Helicobacter pylori and Gastric Cancer: A New Paradigm For Inflammation-Associated Epithelial Cancers. Gastroenterology 2005; 128:1567-78.
14. El-Omar E M, et al. Interleukin-1 polymorphism associated with increased risk of gastric cancer. Nature 2000;404:398-402.
15. Figueiredo C, Machado J C, Pharoah P, Seruca R, Sousa S, Carvalho R, Capelinha A F, Quint W, Caldas C, van Doom L J, Carneiro F, Sobrinho-Simoes M. Helicobacter pylori and interleukin 1 genotyping: an opportunity to identify high-risk individuals for gastric carcinoma. J Natl Cancer Inst. 2002;94:1680-7.
16. Basso D, Scrigner M, Toma A, Navaglia F, Di Mario F, Rugge M, Plebani M. Helicobacter pylori infection enhances mucosal interleukin-1 beta, interleukin-6, and the soluble receptor of interleukin-2. Int J Clin Lab Res 1996;26:207-210
17. Beales I L, Calam J. Interleukin 1 beta and tumour necrosis factor alpha inhibit acid secretion in cultured rabbit parietal cells by multiple pathways. Gut 1998;42:227-34
18. Takashima M, et al. Effects of Helicobacter pylori infection on gastric acid secretion and serum gastrin levels in Mongolian gerbils. Gut 2001;48:765-73.
19. El-Omar E M. The importance of interleukin 1β in Helicoobacterpylori associated disease. Gut 2001;48:743-747.
20. Chen A, Li C N, Hsu P I, Lai K H, Tseng H H, Hsu P N, Lo G H, Lo C C, Lin C K, Hwang I R, Yamaoka Y, Chen H C. Risks of interleukin-1 genetic polymorphisms and Helicobacter pylori infection in the development of gastric cancer. Aliment Pharmacol Ther. 2004;20:203-11.
21. Machado J C, Figueiredo C, Canedo P, Pharoah P, Carvalho R, Nabais S, Castro Alves C, Campos M L, Van Doom L J, Caldas C, Seruca R, Carneiro F, Sobrinho-Simoes M. A proinflammatory genetic profile increases the risk for chronic atrophic gastritis and gastric carcinoma. Gastroenterology. 2003;125:364-71.
22. El-Omar E M, Rabkin C S, Gammon M D, Vaughan T L, Risch H A, Schoenberg J B, Stanford J L, Mayne S T, Goedert J, Blot W J, Fraumeni J F Jr, Chow W H. Increased risk of noncardia gastric cancer associated with proinflammatory cytokine gene polymorphisms. Gastroenterology. 2003;124:1193-201.
23. Lorenz R G, Gordon J I. Use of transgenic mice to study regulation of gene expression in the parietal cell lineage of gastric units. J Biol Chem 1993; 268:26559-70.
24. Bjorkdahl O, Akerblad P, Gjorloff-Wingren A, Leanderson T, Dohlsten M. Lymphoid hyperplasia in transgenic mice over-expressing a secreted form of the human interleukin-1beta gene product. Immunology 1999;96(1):128-37.
25. Fox J G, Beck P, Dangler C A, Whary M T, Wang T C, Shi H N, Nagler-Anderson C. Concurrent enteric helminth infection modulates inflammation and gastric immune responses and reduces helicobacter-induced gastric atrophy. Nat Med 2000;6:536-42.
26. Fox J G, Sheppard B J, Dangler C A, Whary M T, Ihrig M, Wang T C. Germ-line p53-targeted disruption inhibits helicobacter-induced premalignant lesions and invasive gastric carcinoma through down-regulation of Th1 proinflammatory responses. Cancer Res 2002;62:696-702.
27. Uemura N, Okamoto S, Yamamoto S, Matsumura N, Yamaguchi S, Yamakido M, Taniyama K, Sasaki N, Schlemper R J. Helicobacter pylori infection and the development of gastric cancer. N Engl J Med 2001;345:784-789.
28. Uedo N, Tatsuta M, Iishi H, Baba M, Yano H, Ishihara R, Higashino K, Ishiguro S. Enhancement by interleukin-1 beta of gastric carcinogenesis induced by N-methyl-N′-nitro-N-nitrosoguanidine in Wistar rats: a possible mechanism for Helicobacter pylori-associated gastric carcinogenesis. Cancer Lett. 2003; 198:161-8.
29. Furuta T, Shirai N, Sugimoto M. Controversy in polymorphisms of interleukin-1beta in gastric cancer risks. J Gastroenterol. 2004;39:501-3.
30. Lee K A, Ki C S, Kim H J, Sohn K M, Kim J W, Kang W K, Rhee J C, Song S Y, Sohn T S. Novel interleukin 1beta polymorphism increased the risk of gastric cancer in a Korean population. J Gastroenterol. 2004;39:429-33
31. Zheng L, Wang L, Ajani J, Xie K. Molecular basis of gastric cancer development and progression. Gastric Cancer. 2004;7:61-77.
32. Nardone G, Rocco A, Malfertheiner P. Review article: helicobacter pylori and molecular events in precancerous gastric lesions. Aliment Pharmacol Ther 2004;20:261-70.
33. Uemura N, Okamoto S, Yamamoto S, Matsumura N, Yamaguchi S, Yamakido M, Taniyama K, Sasaki N, Schlemper Helicobacter pylori infection and the development of gastric cancer. R J. N Engl J Med. 2001;345:784-9
34. Macarthur M, Hold G L, El-Omar E M. Inflammation and Cancer II. Role of chronic inflammation and cytokine gene polymorphisms in the pathogenesis of gastrointestinal malignancy. Am J Physiol Gastrointest Liver Physiol. 2004;286:G515-20.
35. Kuwano T, Nakao S, Yamamoto H, Tsuneyoshi M, Yamamoto T, Kuwano M, Ono M. Cyclooxygenase 2 is a key enzyme for inflammatory cytokine-induced angiogenesis. FASEB J 2004 ;8:300-10.
36. Tsuzaki M, Guyton G, Garrett W, Archambault J M, Herzog W, Almekinders L, Bynum D, Yang X, Banes A J. IL-1 beta induces COX2, MMP-1, -3 and -13, ADAMTS-4, IL-1 beta and IL-6 in human tendon cells. J Orthop Res 2003;21 :256-64
37. Eaton K A, Mefford M, and Thevenot T. The role of T cell subsets and cytokines in the pathogenesis of Helicoobacterpylori gastritis in mice. J Immunol 2001;166: 7456-7461
38. Eaton K A, Ringler S R, Danon S J. Murine splenocytes induce severe gastritis and delayed-type hypersensitivity and suppress bacterial colonization in Helicobacter pylori-infected SCID mice. Infect Immun. 1999 ;67:4594-602.
39. Dinarello C A. Biological basis for interleukin-1 in disease. Blood 1996;87:2095-2147
40. Yasuda M, Nakano K, Yasumoto K, Tanaka Y. CD44: functional relevance to inflammation and malignancy. Histol Histopathol. 2002;17(3):945-50.
41. Schmits R. CD44 Regulates Hematopoietic Progenitor Distribution, ranuloma Formation, and Tumorigenicity. Blood, Sep. 15, 1997; 90(6): 2217-2233.
42. Balkwill F, Mantovani A. Inflammation and cancer: back to Virchow? Lancet. Feb. 17 2001;357(9255):539-45. Review.
43. Greten F R, Eckmann L, Greten T F, Park J M, Li Z W, Egan L J, Kagnoff M F, Karin M. IKKbeta links inflammation and tumorigenesis in a mouse model of colitis-associated cancer. Cell. Aug. 6, 2004;118(3):285-96.
44. Maeda S, Kamata H, Luo J L, Leffert H, Karin M. IKKbeta couples hepatocyte death to cytokine-driven compensatory proliferation that promotes chemical hepatocarcinogenesis. Cell. Jul. 1, 2005;121(7):977-90.
45. Ohno S, Inagawa H, Dhar D K, Fujii T, Ueda S, Tachibana M, Suzuki N, Inoue M, Soma G, Nagasue N. The degree of macrophage infiltration into the cancer cell nest is a significant predictor of survival in gastric cancer patients. Anticancer Res. November-December 2003;23(6D):5015-22.
46. Fox J G, Wang T C. Helicobacter pylori—not a good bug after all! N Engl J Med. Sep. 13, 2001;345(11):784-9.
47. Barber M D, Powell J J, Lynch S F, et al: A polymorphism of the interleukin-1 beta gene influences survival in pancreatic cancer. Br J Cancer 83:1443-1447 (2000).
48. Howell W M, Turner S J, Theaker J M, et al: Cytokine gene single nucleotide polymorphisms and susceptibility to and prognosis in cutaneous malignant melanoma. Eur J Immunogenet 30:409-414 (2003).
49. Zienolddiny S, Ryberg D, Maggini V, Skaug V, Canzian F, Haugen A. Polymorphisms of the interleukin-1 beta gene are associated with increased risk of non-small cell lung cancer. Int J Cancer. 109:353-356 (2004).
50. Wang Y, Kato N, Hoshida Y, Yoshida H, Taniguchi H, Goto T, Moriyama M, Otsuka M, Shiina S, Shiratori Y, Ito Y, Omata M. Interleukin-1beta gene polymorphisms associated with hepatocellular carcinoma in hepatitis C virus infection. Hepatology. 37:65-71 (2003).
51. Lin R M, Francois F, Olivares A, Williams R, Huang G, Poles M A, Perez-Perez G, IL-1beta-511 Polymorphism Is Associated With Increased Risk Of Colonic Adenomas in An Ethnically Diverse Population. Gastroenterology 128 (4): A9 Suppl. 2, 2005
52. Furuta T, Shirai N, Sugimoto M. Controversy in polymorphisms of interleukin-1beta in gastric cancer risks. J Gastroenterol. 39:501-503 (2004).
53. Lee K A, Ki C S, Kim H J, Sohn K M, Kim J W, Kang W K, Rhee J C, Song S Y, Sohn T S. Novel interleukin 1beta polymorphism increased the risk of gastric cancer in a Korean population. J Gastroenterol. 39:429-433 (2004).
54. Yeo A E, Tanaka Y, Furuta T. Interleukin lbeta gene polymorphism and hepatitis C virus-related hepatocellular carcinoma. Interleukin 1beta gene polymorphism and hepatitis C virus-related hepatocellular carcinoma. Hepatology. 38:267-268; 2003
55. Hwang I R, Kodama T, Kikuchi S, Sakai K, Peterson L E, Graham D Y, Yamaoka Y. Effect of interleukin 1 polymorphisms on gastric mucosal interleukin lbeta production in Helicobacter pylori infection. Gastroenterology. December 2002;123(6): 1793-803.
56. Machado J C, Figueiredo C, Canedo P, Pharoah P, Carvalho R, Nabais S, Castro Alves C, Campos M L, Van Doom L J, Caldas C, Seruca R, Cameiro F, Sobrinho-Simoes M. A proinflammatory genetic profile increases the risk for chronic atrophic gastritis and gastric carcinoma. Gastroenterology. August 2003;125(2):364-71.
57. Takashima M, Furuta T, Hanai H, Sugimura H, Kaneko E. Effects of Helicobacter pylori infection on gastric acid secretion and serum gastrin levels in Mongolian gerbils. Gut. June 2001;48(6):765-73.
58. Mandell L, Moran A P, Cocchiarella A, Houghton J, Taylor N, Fox J G, Wang T C, Kurt-Jones E A. Intact gram-negative Helicobacter pylori, Helicobacter felis, and Helicobacter hepaticus bacteria activate innate immunity via toll-like receptor 2 but not toll-like receptor 4. Infect Immun. November 2004;72(11):6446-54.

What is claimed is:

1. A transgenic non-human mammal whose somatic and germ cells comprise (a) a DNA segment encoding a human cytokine operably linked to a DNA segment encoding a secretory signal sequence; and (b) a constitutively active tissue-specific promoter wherein (a) is operably linked to (b).

2. The mammal of claim 1, wherein the tissue for which the promoter is specific comprises breast tissue, colon tissue, pancreas tissue, lung tissue, ovary tissue, cervical tissue, uterine tissue, bone tissue, stomach tissue, gastric tissue, testicular tissue, prostate tissue, skin tissue, esophagus tissue, liver tissue, kidney tissue, bladder tissue, or any combination thereof.

3. The mammal of claim 1, wherein the cytokine comprises interleukin-1 beta, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

4. The mammal of claim 1, wherein the linked DNA segments are integrated into the mammal's genome.

5. A transgenic non-human mammal whose somatic and germ cells comprise
(a) a DNA segment encoding human interleukin-1β operably linked to a DNA segment encoding a secretory signal sequence; and
(b) a constitutively active parietal cell-specific promoter wherein (a) is operably linked to (b).

6. The mammal of claim 5, wherein expression of the DNA segment results in gastritis, dysplasia, spontaneous development of gastric cancer, or any combination thereof in the mammal.

7. The mammal of claim 5, wherein the promoter comprises a mouse H/K-ATPase promoter, or a functional fragment thereof

8. A transgenic non-human mammal whose somatic and germ cells comprise
(a) a DNA segment encoding human interleukin-1β operably linked to a DNA segment encoding a secretory signal sequence; and
(b) a constitutively active pancreas-specific promoter
wherein (a) is operably linked to (b).

9. The mammal of claim 8, wherein expression of the DNA segment results in pancreatic intraepithelial neoplasia, spontaneous development of pancreatic cancer, or a combination thereof in the mammal.

10. The mammal of claim 8, wherein the promoter comprises a rat elastase promoter, or a functional fragment thereof.

11. The mammal of claim 5 or 8, wherein the secretory signal sequence comprises a signal sequence from an IL-1 receptor antagonist gene, or a fragment thereof.

12. The mammal of claim 5 or 8, wherein the mammal is a mouse.

13. A transgenic non-human mammal whose somatic and germ cells comprise a DNA segment encoding a human cytokine operably linked to a constitutively active H/K-ATPase promoter, K19 promoter, TFF1 promoter, TFF2 promoter, FOXa3/HNF3γ promoter, or a functional fragment thereof, wherein expression of the DNA segment results in spontaneous development of cancer in the stomach of the mammal.

14. A transgenic non-human mammal whose somatic and germ cells comprise a DNA segment encoding a human cytokine operably linked to a constitutively active Clara cell secretory protein promoter, surfactant protein C promoter, or a fragment thereof, wherein expression of the DNA segment results in spontaneous development of cancer in the lungs of the mammal.

15. A transgenic non-human mammal whose somatic and germ cells comprise a DNA segment encoding a human cytokine operably linked to a constitutively active mammary tumor virus, whey acidic protein promoter, or a fragment thereof, wherein expression of the DNA segment results in spontaneous development of cancer in a breast of the mammal.

16. A transgenic non-human mammal whose somatic and germ cells comprise a DNA segment encoding a human cytokine operably linked to a constitutively active Pdx-1 promoter, insulin promoter, phosphoglycerate kinase promoter, elastase promoter, or a fragment thereof, wherein expression of the DNA segment results in spontaneous development of cancer in the pancreas of the mammal.

17. A transgenic non-human mammal whose somatic and germ cells comprise a DNA segment encoding a human cytokine operably linked to a constitutively active keratin promoter, K14 promoter, or a fragment thereof, wherein expression of the DNA segment results in spontaneous development of cancer in the skin of the mammal.

18. A transgenic non-human mammal whose somatic and germ cells comprise a DNA segment encoding a human cytokine operably linked to a constitutively active EBV ED-L2 promoter, or a fragment thereof, wherein expression of the DNA segment results in spontaneous development of cancer in the esophagus of the mammal.

19. A transgenic non-human mammal whose somatic and germ cells comprise a DNA segment encoding a human cytokine operably linked to a constitutively active major urinary protein promoter, albumin promoter, or a fragment thereof, wherein expression of the DNA segment results in spontaneous development of cancer in the liver of the mammal.

20. A transgenic non-human mammal whose somatic and germ cells comprise a DNA segment encoding a human cytokine operably linked to a constitutively active villin promoter, FABP-TS4 promoter, or a fragment thereof, wherein expression of the DNA segment results in spontaneous development of cancer in the colon of the mammal.

21. A transgenic non-human mammal whose somatic and germ cells comprise a DNA segment encoding a human cytokine operably linked to a constitutively active cryptdin-2 promoter, prostate-specific antigen (PSA) promoter, C(3)1 promoter, prostate secretory protein of 94 amino acids (PSP94) promoter, or the probasin promoter, or a fragment thereof, wherein expression of the DNA segment results in spontaneous development of cancer in the prostate of the mammal.

22. A transgenic non-human mammal whose somatic and germ cells comprise a DNA segment encoding a human cytokine operably linked to a constitutively active ovarian-specific promoter (OSP-1), or a fragment thereof, wherein expression of the DNA segment results in spontaneous development of cancer in one or both ovaries of the mammal.

23. A transgenic non-human mammal whose somatic and germ cells comprise a DNA segment encoding a human cytokine operably linked to a constitutively active uromodulin promoter, Tamm-Horsfall protein (THP) promoter, or type 1 gamma-glutamyl transpeptidase promoter, or a fragment thereof, wherein expression of the DNA segment results in spontaneous development of cancer in one or both kidneys of the mammal.

24. A transgenic non-human mammal whose somatic and germ cells comprise a DNA segment encoding a human cytokine operably linked to a constitutively active uroplakin promoter or urohingin promoter, or a fragment thereof, wherein expression of the DNA segment results in spontaneous development of cancer in the bladder of the mammal.

25. A transgenic non-human mammal whose somatic and germ cells comprise a DNA segment encoding a human cytokine operably linked to a constitutively active uteroglobin promoter, or a fragment thereof, wherein expression of the DNA segment results in spontaneous development of cancer in the uterus of the mammal.

26. The mammal of any of claims 1-25, wherein the cytokine comprises an inflammatory cytokine.

27. The mammal of any of claims 1-25, wherein the cytokine comprises a secreted form of human interleukin-1.

28. The mammal of any of claims 1-25, wherein the cytokine comprises TNF-α, IL-1, IL-2, IL-3, IL-4, IL-5, IL-6, IL-7, IL-8, IL-9, IL-10, IL-11, IL-12, IL-13, IL-14, IL-15, IL-16, IL-18, G-CSF, GM-CSF, TNF-β, TGF-β, IFN-γ, IFN-α/β, SDF-1/CXCL12, MIP1β/CCL3, MCP-1/CCL2, SCF, or any combination thereof.

29. A cell from the non-human transgenic mammal of any of claims 1-25.

30. A method for identifying whether a test compound is capable of treating cancer, the method comprising
(a) administering an effective amount of a test compound to a transgenic non-human mammal of any of claims 1-25;
(b) measuring progression of cancer in the transgenic non-human mammal of (a); and
(c) comparing the progression of cancer measured in (b) to progression of cancer measured in a sibling of the transgenic non-human mammal, wherein the sibling was not administered the test compound, and wherein a decrease in progression of cancer in the non-human mammal of (a) compared to (b) indicates that the test compound is capable of treating cancer.

31. The method of claim 30, wherein the transgenic non-human mammal has cancer.

32. The method of claim 30, wherein a decrease comprises an arrest, delay or reversal in progression of cancer.

33. The method of claim 30, wherein the measuring comprises a histological assessment, an assessment of alterations in the mammal's weight and activity, non-invasive imaging, an assessment of serum biomarkers, or any combination thereof.

34. A method for identifying whether a test compound is capable of preventing or delaying the development of cancer, the method comprising
(a) administering an effective amount of a test compound to a transgenic non-human mammal of any of claims 1-25, wherein the transgenic non-human mammal does not have cancer;
(b) measuring development of cancer in the transgenic non-human mammal of (a);
(c) comparing the development of cancer measured in (b) to development of cancer measured in a sibling of the transgenic non-human mammal, wherein the sibling was not administered the test compound, and wherein inhibition of or a delay in the development of cancer in the non-human mammal of (a) compared to (b) indicates that the test compound is capable of preventing or delaying the development of cancer.

35. A method for identifying whether a test compound is a carcinogen, the method comprising
(a) administering to a transgenic non-human mammal of any of claims 1-25 or exposing a transgenic non-human mammal of any of claims 1-25 to an effective amount of a test compound, wherein the transgenic non-human mammal does not have cancer;
(b) measuring development of cancer in the transgenic non-human mammal of (a);
(c) comparing the development of cancer measured in (b) to development of cancer measured in a sibling of the transgenic non-human mammal, wherein the sibling was not administered the test compound or exposed to the test compound, and wherein earlier development of cancer in the non-human mammal of (a) compared to (b) indicates that the test compound is a carcinogen.

36. The method of any of claims 30-35, wherein the cancer comprises a breast cancer, a colon cancer, a pancreatic cancer, a lung cancer, an ovarian cancer, a cervical cancer, a uterine cancer, a bone cancer, a stomach cancer, a gastric cancer, a testicular cancer, a prostate cancer, a skin cancer, an esophageal cancer, a liver cancer, a kidney cancer, a bladder cancer, a lymphoma, or any combination thereof.

37. A nucleic acid comprising:
(a) a tissue-specific promoter operably linked to a DNA segment encoding a secreted human cytokine, or a fragment thereof; and
(b) a polyadenylation signal,
wherein (a) and (b) are operably linked and wherein the nucleic acid is capable of producing expression of the cytokine in the specific tissue in a transgenic mammal.


SEQUENCE LISTING (18 sequences)

SEQ ID NO: 1 - DNA, 25 bp, Artificial
  Synthetic primer
tgcgaatctccgaccaccactacag

SEQ ID NO: 2 - DNA, 28 bp, Artificial
  Synthetic primer
tggaggtggagagctttcagttcatatg

SEQ ID NO: 3 - DNA, 24 bp, Artificial
  Synthetic primer
catggctgtttctggctgttactg

SEQ ID NO: 4 - DNA, 24 bp, Artificial
  Synthetic primer
gttgctgatggcctgattgtcttt

SEQ ID NO: 5 - DNA, 22 bp, Artificial
  Synthetic primer
tggcccagaccctcacactcag

SEQ ID NO: 6 - DNA, 20 bp, Artificial
  Synthetic primer
acccatcggctggcaccact

SEQ ID NO: 7 - DNA, 24 bp, Artificial
  Synthetic primer
ggagaaccaagcaacgacaaaata

SEQ ID NO: 8 - DNA, 23 bp, Artificial
  Synthetic primer
tggggaactctgcagactcaaac

SEQ ID NO: 9 - DNA, 20 bp, Artificial
  Synthetic primer
ctgcctgcccccacagaaga

SEQ ID NO: 10 - DNA, 22 bp, Artificial
  Synthetic primer
gcgcagagtctcgccattatga

SEQ ID NO: 11 - DNA, 22 bp, Artificial
  Synthetic primer
gttttctgcaagtgcatcatcg

SEQ ID NO: 12 - DNA, 22 bp, Artificial
  Synthetic primer
gttttctgcaagtgcatcatcg

SEQ ID NO: 13 - DNA, 22 bp, Artificial
  Synthetic primer
atcggcattttgaacgaggtca

SEQ ID NO: 14 - DNA, 23 bp, Artificial
  Synthetic primer
catcgaaaagcccgaaagagtct

SEQ ID NO: 15 - DNA, 23 bp, Artificial
  Synthetic primer
cctagagctgcggactgccttca

SEQ ID NO: 16 - DNA, 22 bp, Artificial
  Synthetic primer
cagccgcatcctgagggtcttc

SEQ ID NO: 17 - DNA, 20 bp, Artificial
  Synthetic primer
ggagaaacctgccaagtatg

SEQ ID NO: 18 - DNA, 20 bp, Artificial
  Synthetic primer
tgggagttgctgttgaagtc